(19) United States
(12) Patent Application Publication
(10) Pub. No.: US 2017/0340563 A1
(43) Pub. Date: Nov. 30, 2017

(54) Methods for the Preparation of Liposomes Comprising Drugs

(71) Applicant: CuriRx Inc., Wilmington, MA (US)

(72) Inventors: Indu JAVERI, North Andover, MA (US); Kaliappanadar NELLAIAPPAN, Lexington, MA (US)

(21) Appl. No.: 15/593,586

(22) Filed: May 12, 2017

Related U.S. Application Data
(63) Continuation-in-part of application No. 14/088,657, filed on Nov. 25, 2013, now Pat. No. 9,655,846.
Application No. 14/088,657 is a continuation of application No. 12/889,305, filed on Sep. 23, 2010, now Pat. No. 8,591,942.
(60) Provisional application No. 61/245,185, filed on Sep. 23, 2009.

(51) Int. Cl. A61K 9/127; A61K 31/337; A61K 9/00; A61K 9/19

(52) U.S. Cl. CPC A61K 9/127; A61K 9/1278; A61K 31/337; A61K 9/0095; A61K 9/19

(57) ABSTRACT

Provided herein are methods for preparing liposomes comprising increased concentration of hydrophobic therapeutic agents and improved stability, and uses thereof. In certain embodiments, liposomes are prepared without using heat, organic solvents, proteins, and/or inorganic salts.

### CROSS REFERENCE TO RELATED APPLICATIONS

This application is a continuation-in-part of U.S. application Ser. No. 14/088,657, filed Nov. 25, 2013, which is a continuation of U.S. application Ser. No. 12/889,305, filed Sep. 23, 2010, now U.S. Pat. No. 8,591,942, which claims the benefit under 35 U.S.C. §119 of U.S. Provisional Patent Application No. 61/245,185, filed Sep. 23, 2009. The disclosures of the aforementioned applications are incorporated by reference in their entireties herein.

### BACKGROUND

The bioavailability of a pharmaceutical drug depends largely in part on the solubility and stability of the drug. Many methods have been employed to improve bioavailability of a drug, including, but not limited to, pH adjustment, associating the drug in micelles of detergents, solubilization in an organic solvent, complexation with cyclodextrin or other polymers, and encapsulating the drug in a liposome bilayer (Strickley, R. G., Pharmaceutical Research, No. 21, 2004: 201-230). Either the drug itself or the excipients used to solubilize the drug may have side effects such as allergic reaction or hemolysis.

It is known that the solvents (e.g., ethanol, propylene glycol, polyethylene glycol, dimethylacetamide, dimethylsulfoxide (“DMSO”)), complexing agents (for example, nicotinamide), and surfactants (for example, sodium oleate) are hemolytic and are therefore undesirable for use in injectable solutions. Other limitations to using organic solvents in injectable products include precipitation, pain, and inflammation upon injection.

Liposomes are microscopic lipid vesicles that are composed of a central aqueous cavity surrounded by a lipid membrane formed by concentric bilayer(s) (lamellas). Liposomes are able to incorporate hydrophilic substances (in the aqueous interior) or hydrophobic substances (in the lipid membrane). Liposomes can be unilamellar vesicles (“UMV”), having a single lipid bilayer, or multilamellar vesicles (“MLV”), having a series of lipid bilayers (also referred to as “oligolamellar vesicles”). The multilamellar vesicles typically range in size from 0.2 μm to 10 μm in diameter. See e.g., WO 98/006882. Although anti-hemolytic measures are commonly taken in formulations, maintaining a sufficient amount of liposome in formulation may not be feasible due to the incompatibility of the liposome with an excipient, or the instability of the liposome in the formulation. Further, reconstituting lyophilized formulations containing hydrophobic drugs is often difficult. This is the case, for example, in the reconstitution of docetaxel, sodium oleate, and liposomes. Moreover, liposomes are not stable in formulations containing concentrated organic solvents.

Unilamellar vesicles with a diameter of less than 0.2 μm (e.g. between 0.02 and 0.2 μm) are commonly known as small unilamellar vesicles (“SUV”). Unilamellar vesicles with a diameter greater than 0.45 μm (in some cases greater than 1 μm) are commonly known as large unilamellar vesicles (“LUV”).

The bilayer(s) of liposomes most often comprise phospholipids, but may also comprise lipids including but not limited to fatty acids, fatty acid salts and/or fatty alcohols. The properties of the liposomes depend, among other factors, on the nature of the constituents. Consequently, if liposomes with certain characteristics are to be obtained, the charge of its polar group and/or the length and the degree of saturation of its fatty acid chains must be taken into account.

In addition, the properties of liposomes may be modified, e.g., to incorporate cholesterol and other lipids into the membrane, change the number of lipidic bilayers, or covalently join natural molecules (e.g., proteins, polysaccharides, glycolipids, antibodies, enzymes) or synthetic molecules (e.g., polyethyl glycol) to the surface. There are numerous combinations of phospholipids, optionally with other lipids or cholesterol, in an aqueous medium to obtain liposomes. Depending on the method of preparation and the lipids used, it is possible to obtain vesicles of different sizes, structures, and properties.

Another important parameter to consider with respect to the formation of liposomes is the rigidity of the lipid bilayer. The hydrated lipid that forms part of the bilayer may be in either a liquid-crystalline (fluid) or gel state. As the temperature increases, the gel state is converted into the liquid-crystalline state. This occurs at a temperature known as the transition temperature (Tc), which is specific to each lipid. The Tc is directly proportional to chain length and inversely proportional to the degree of unsaturation of the fatty acids and depends on the nature of the polar group.

Despite this, common methods in the preparation of lipid vesicles, such as liposomes, comprise evaporating an organic solvent in which the lipids are dissolved and then dispersed in an optionally buffered aqueous solution. One exemplary method, known as the Bangham method, was originally described in Bangham et al., J. Mol. Biol., 11:238-252 (1965). Variations of the Bangham method are known by those skilled in the art, some of which are described below.

Hydration of a Thin Lipidic Layer.

Starting with the organic solution of the constituent lipids of the bilayer, a lipidic film is prepared through removal of organic solvent, which can be achieved by means of evaporation (e.g., at reduced pressure in a rotary evaporator) or by lyophilization. The dry lipidic film obtained is hydrated by adding an aqueous solution and agitating the mixture at temperatures above the Tc.

Reverse-Phase Evaporation.

Starting with the organic solution of the constituent lipids of the bilayer, a lipidic film is prepared through removal of the organic solvent. The system is purged with nitrogen and the lipids are re-dissolved in a second organic solution, usually constituted by diethyl ether and/or isopropyl ether. The aqueous phase is added to the re-dissolved lipids. The system is maintained under continuous nitrogen. A gel is formed by removing the second organic solvent.

Solvent Injection.

The lipids, dissolved in an organic solvent, are injected slowly into an aqueous solution. The organic solvent used is often a water-miscible solvent, and the aqueous solution may be warmed.

Additional methods for the preparation of multilamellar vesicles can be found, e.g., in Szoka and Papandjopoulos, Ann. Rev. Biophys. Bioeng., 2: 467-508 (1980), and Dousset and Douste-Blazy, Les Liposomes, Puisieux and Delattre, Editors, Tecniques et Documentation Lavoisier, Paris, pp. 1-73 (1985).

Further, when the incorporation of more than one lipid is desired, the lipids should remain homogeneously distributed in the liposomal vesicles. Traditionally, this is achieved by previously dissolving the lipids in an organic solvent and using the resulting organic solvent for preparing the liposomes.

U.S. Pat. No. 4,508,703 describes a method for obtaining powdery mixtures of at least one amphiphilic lipid and, optionally, at least one component of a hydrophobic or partially hydrophobic nature, a method which includes dissolving the components of the mixture in at least one organic solvent and atomizing the obtained solution into an inert gas. The method permits the preparation of lipidic mixtures which can be easily dispersed in an aqueous medium but does not avoid the use of organic solvents.

WO 92/10166 describes a method for preparing liposomes with an elevated encapsulation capacity. The method permits the use of mixtures of lipids; however, the mixture is obtained by means of previous dissolution of the lipids in an organic solvent and subsequent evaporation. In addition, the contact between the lipids and the aqueous solution of active agent is carried out at a temperature above the Tc.

Moreover, it is reported that, where liposomes are made without using organic solvents, other manipulations, which may result in formulations with certain unfavorable characteristics, are generally required. For example, U.S. Pat. App. Pub. No. 2008/0274172 describes methods of preparing liposomes containing at least two phospholipids without using organic solvents. However temperatures above the Tc were used to obtain stable liposomes from aqueous solutions containing inorganic salts. Consequently, existing methods for preparing liposomes utilize organic solvents, protein, inorganic salts, and/or heat. Due to their toxicity and flammability, organic solvents are undesirable in the preparation of liposomes for pharmaceutical, cosmetic and other uses. Moreover, the use of organic solvents and proteins has negative repercussions in terms of production costs, safety, work hygiene and the environment. Similarly, the use of heat in the preparation of liposomes is undesirable in terms of production costs, safety, and the environment.

The use of inorganic salts in the preparation of liposomes is undesirable as the introduction of inorganic salts increases the size of the liposome and/or results in a more turbid formulation. See e.g. Castile et al., International Journal of Pharmaceutics, 1999, vol. 188, issue 1, pp. 87-95. Thus, there is a need for a method for preparing liposomes without the use of undesirable agents and procedures.

### SUMMARY

Provided herein are methods for incorporating a hydrophobic therapeutic agent into liposomes. In certain embodiments liposomes are prepared without using heat, organic solvents, proteins, and/or inorganic salts in the process.

In one embodiment, a method is provided for incorporating a hydrophobic therapeutic agent into preformed liposomes, the method comprising:

(a) providing (i) a liposome suspension comprising a plurality of preformed liposomes suspended in an aqueous medium, the liposomes comprising lipid forming a lipid bilayer phase, and (ii) a solid form of a hydrophobic therapeutic agent;

(b) adding the solid form of the hydrophobic therapeutic agent to the liposome suspension, thereby forming a liposome-drug suspension; and

(c) homogenizing the liposome-drug suspension;

whereby the hydrophobic therapeutic agent is incorporated into the lipid bilayer phase; and

wherein step (c) is performed at a temperature at or below ambient temperature.

In certain embodiments, steps (b) and/or (c) are performed in the absence of solvent. In certain embodiments, steps (b) and/or (c) are performed in the absence of surfactant.

In certain embodiments, the hydrophobic therapeutic agent is a small molecule drug or an antibody. In certain embodiments, at least about 80% of the hydrophobic therapeutic agent is bound to the lipid bilayer phase after step (c).

In certain embodiments, the molar ratio of therapeutic agent to liposomal lipid is at least about 1:10.

In certain embodiments, homogenization of the liposome-drug suspension in step (c) is performed by microfluidization, sonication, extrusion, freeze-thaw, or a combination thereof.

In certain embodiments, the therapeutic agent does not contact solvent during steps (b) and (c), and does not contact the liposomal lipid prior to the formation of the liposomes.

In certain embodiments, the liposomes are essentially unilamellar after step (c). In certain embodiments, the liposomes have a diameter of about 100 nm or less after step (c). In certain embodiments, the liposomes have a diameter of about 50 nm or less after step (c).

In certain embodiments, the liposome suspension provided in step (a) includes an additional therapeutic agent present in the aqueous medium and/or the liposomes.

In certain embodiments, the composition of the aqueous medium inside and outside the liposomes is identical in steps (a), (b), and (c).

In certain embodiments, the liposomal lipid includes not more than 20% saturated fatty acids. In certain embodiments, the liposomal lipid includes L-α-phosphatidylcholine.

In certain embodiments, the method further includes, after step (c), performing sterile filtration, lyophilization, or lyophilization and reconstitution with an aqueous medium.

In certain embodiments, the therapeutic agent remains bound to the lipid bilayer phase of the liposomes after step (c) for at least 2 months upon storage in the aqueous medium at about 4° C. In certain embodiments, the therapeutic agent remains associated with the lipid bilayer phase after step (c) followed by lyophilization and storage for at least 1 year at ambient temperature and reconstitution in an aqueous medium.

In certain embodiments, average liposome size after step (c) remains less than about 100 nm for at least 2 months upon storage in the aqueous medium at about 4° C. In certain embodiments, after step (c) followed by lyophilization, storage for at least 1 year at ambient temperature, and reconstitution in an aqueous medium, the average liposome size remains less than about 100 nm.

In certain embodiments, the ambient temperature is from about 15° C. to about 35° C.

In certain embodiments, the lipid bilayer phase is liquid crystalline at said temperature.

In certain embodiments, the lipid concentration of the liposome-drug suspension in steps (b) and (c) is from about 1 to about 7% by weight.

In certain embodiments, the solid hydrophobic therapeutic agent added in step (b) provides a total concentration of the agent in the liposome-drug suspension of from about 1 to about 20 mg/mL.

In another embodiment, a method is provided for incorporating a hydrophobic therapeutic agent into preformed liposomes, the method comprising the steps of:
(a) providing (i) a liposome suspension comprising a plurality of preformed liposomes suspended in an aqueous medium, the liposomes comprising lipid forming a lipid bilayer phase, and (ii) a therapeutic agent concentrate comprising a hydrophobic therapeutic agent dissolved in a liquid medium comprising or consisting of solvent;
(b) adding the therapeutic agent concentrate to the liposome suspension to form a liposome-drug suspension, wherein the total concentration of solvent in the liposome-drug suspension is not more than 10 weight percent; and
(c) homogenizing the liposome-drug suspension;

whereby the hydrophobic therapeutic agent is incorporated into the lipid bilayer phase; and

wherein step (c) is performed at a temperature at or below ambient temperature.

In certain embodiments, the total concentration of solvent in the liposome-drug suspension in step (b) is not more than about 5 weight percent. In certain embodiments, the solvent is a water miscible organic solvent.

In certain embodiments, the liquid medium further includes water or an aqueous medium.

In certain embodiments, the solvent is selected from the group consisting of alcohols, ketones, ethers, organic acids, organic bases, and mixtures thereof. In certain embodiments, the solvent is selected from the group consisting of ethanol, propanol, isopropanol, butanol, isobutanol, and DMSO.

In certain embodiments, the liquid medium is 100% ethanol.

In certain embodiments, step (c) is performed without the use of surfactant.

In certain embodiments, the hydrophobic therapeutic agent is a small molecule drug or an antibody. In certain embodiments, at least about 80% of the hydrophobic therapeutic agent is bound to the lipid bilayer phase after step (c).

In certain embodiments, the molar ratio of therapeutic agent to liposomal lipid is at least about 1:10.

In certain embodiments, homogenization of the liposome-drug suspension in step (c) is performed by microfluidization, sonication, extrusion, freeze-thaw, or a combination thereof.

In certain embodiments, the therapeutic agent does not contact solvent during steps (b) and (c), and does not contact the liposomal lipid prior to the formation of the liposomes.

In certain embodiments, the liposomes are essentially unilamellar after step (c). In certain embodiments, the liposomes have a diameter of about 100 nm or less after step (c). In certain embodiments, the liposomes have a diameter of about 50 nm or less after step (c).

In certain embodiments, the liposome suspension provided in step (a) includes an additional therapeutic agent present in the aqueous medium and/or the liposomes.

In certain embodiments, the composition of the aqueous medium inside and outside the liposomes is identical in steps (a), (b), and (c).

In certain embodiments, the liposomal lipid includes not more than 20% saturated fatty acids. In certain embodiments, the liposomal lipid includes L-α-phosphatidylcholine.

In certain embodiments, the method further includes, after step (c), performing sterile filtration, lyophilization, or lyophilization and reconstitution with an aqueous medium.

In certain embodiments, the therapeutic agent remains bound to the lipid bilayer phase of the liposomes after step (c) for at least 2 months upon storage in the aqueous medium at about 4° C. In certain embodiments, the therapeutic agent remains associated with the lipid bilayer phase after step (c) followed by lyophilization and storage for at least 1 year at ambient temperature and reconstitution in an aqueous medium.

In certain embodiments, the homogenized liposome suspension from (c) is lyophilized, whereby the solvent is removed. Preferably at least 95%, at least 97%, at least 98%, at least 99% or essentially 100% of the solvent is removed.

In certain embodiments, average liposome size after step (c) remains less than about 100 nm for at least 2 months upon storage in the aqueous medium at about 4° C. In certain embodiments, after step (c) followed by lyophilization, storage for at least 1 year at ambient temperature, and reconstitution in an aqueous medium, the average liposome size remains less than about 100 nm.

In certain embodiments, the ambient temperature is from about 15° C. to about 35° C.

In certain embodiments, the lipid bilayer phase is liquid crystalline at said temperature.

In certain embodiments, the lipid concentration of the liposome-drug suspension in steps (b) and (c) is from about 1 to about 7% by weight.

In certain embodiments, the solid hydrophobic therapeutic agent added in step (b) provides a total concentration of the agent in the liposome-drug suspension of from about 1 to about 20 mg/mL.

The invention also can be summarized with the following listing of embodiments.

1. A method of incorporating a hydrophobic therapeutic agent into preformed liposomes, the method comprising the steps of:

(a) providing (i) a liposome suspension comprising a plurality of preformed liposomes suspended in an aqueous medium, the liposomes comprising lipid forming a lipid bilayer phase, and (ii) a solid form of a hydrophobic therapeutic agent;

(b) adding the solid form of the hydrophobic therapeutic agent to the liposome suspension, thereby forming a liposome-drug suspension; and

(c) homogenizing the liposome-drug suspension;

whereby the hydrophobic therapeutic agent is incorporated into the lipid bilayer phase; and
wherein step (c) is performed at a temperature at or below ambient temperature.
2. The method of embodiment 1, wherein steps (b) and/or (c) are performed in the absence of solvent.

3. The method of embodiment 1, wherein steps (b) and/or (c) are performed in the absence of surfactant.

4. The method of embodiment 1, wherein the hydrophobic therapeutic agent is a small molecule drug or an antibody.
5. The method of embodiment 1, wherein at least about 80% of the hydrophobic therapeutic agent is bound to the lipid bilayer phase after step (c).
6. The method of embodiment 1, wherein the molar ratio of therapeutic agent to liposomal lipid is at least about 1:10.
7. The method of embodiment 1, wherein homogenization of the liposome-drug suspension in step (c) is performed by microfluidization, sonication, extrusion, freeze-thaw, or a combination thereof.
8. The method of embodiment 1, wherein the therapeutic agent does not contact solvent during steps (b) and (c), and does not contact the liposomal lipid prior to the formation of the liposomes.
9. The method of embodiment 1, wherein the liposomes are essentially unilamellar after step (c).
10. The method of embodiment 1, wherein the liposomes have a diameter of about 100 nm or less after step (c).
11. The method of embodiment 1, wherein the liposomes have a diameter of about 50 nm or less after step (c).
12. The method of embodiment 1, wherein the liposome suspension provided in step (a) comprises an additional therapeutic agent present in the aqueous medium and/or the liposomes.
13. The method of embodiment 1, wherein the composition of the aqueous medium inside and outside the liposomes is identical in steps (a), (b), and (c).
14. The method of embodiment 1, wherein the liposomal lipid comprises not more than 20% saturated fatty acids.
15. The method of embodiment 1, wherein the liposomal lipid comprises L-α-phosphatidylcholine.
16. The method of embodiment 1, wherein the method further comprises, after step (c), performing sterile filtration, lyophilization, or lyophilization and reconstitution with an aqueous medium.
17. The method of embodiment 1, wherein the therapeutic agent remains bound to the lipid bilayer phase of the liposomes after step (c) for at least 2 months upon storage in the aqueous medium at about 4° C.
18. The method of embodiment 1, wherein the therapeutic agent remains associated with the lipid bilayer phase after step (c) followed by lyophilization and storage for at least 1 year at ambient temperature and reconstitution in an aqueous medium.
19. The method of embodiment 1, wherein average liposome size after step (c) remains less than about 100 nm for at least 2 months upon storage in the aqueous medium at about 4° C.
20. The method of embodiment 1, wherein after step (c) followed by lyophilization, storage for at least 1 year at ambient temperature, and reconstitution in an aqueous medium, the average liposome size remains less than about 100 nm.
21. The method of embodiment 1, wherein said temperature is from about 15° C. to about 35° C.
22. The method of embodiment 1, wherein the lipid bilayer phase is liquid crystalline at said temperature.
23. The method of embodiment 1, wherein the lipid concentration of the liposome-drug suspension in steps (b) and (c) is from about 1 to about 7% by weight.
24. The method of embodiment 1, wherein the solid hydrophobic therapeutic agent added in step (b) provides a total concentration of the agent in the liposome-drug suspension of from about 1 to about 20 mg/mL.
25. A method of incorporating a hydrophobic therapeutic agent into preformed liposomes, the method comprising the steps of:

(a) providing (i) a liposome suspension comprising a plurality of preformed liposomes suspended in an aqueous medium, the liposomes comprising lipid forming a lipid bilayer phase, and (ii) a therapeutic agent concentrate comprising a hydrophobic therapeutic agent dissolved in a liquid medium comprising or consisting of solvent;

(b) adding the therapeutic agent concentrate to the liposome suspension to form a liposome-drug suspension, wherein the total concentration of solvent in the liposome-drug suspension is not more than 10 weight percent; and

(c) homogenizing the liposome-drug suspension;

whereby the hydrophobic therapeutic agent is incorporated into the lipid bilayer phase; and
wherein step (c) is performed at a temperature at or below ambient temperature.
26. The method of embodiment 25, wherein the total concentration of solvent in the liposome-drug suspension in step (b) is not more than about 5 weight percent.
27. The method of embodiment 25, wherein the solvent is a water miscible organic solvent.
28. The method of embodiment 25, wherein the liquid medium further comprises water or an aqueous medium.
29. The method of embodiment 25, wherein the solvent is selected from the group consisting of alcohols, ketones, ethers, organic acids, organic bases, and mixtures thereof.
30. The method of embodiment 25, wherein the solvent is selected from the group consisting of ethanol, propanol, isopropanol, butanol, isobutanol, and DMSO.
31. The method of embodiment 25, wherein the liquid medium is 100% ethanol.
32. The method of embodiment 25, wherein step (c) is performed without the use of surfactant.
33. The method of embodiment 25, wherein the hydrophobic therapeutic agent is a small molecule drug or an antibody.
34. The method of embodiment 25, wherein at least about 80% of the hydrophobic therapeutic agent is associated with the lipid bilayer phase after step (c).
35. The method of embodiment 25, wherein the molar ratio of therapeutic agent to lipid is at least about 1:10.
36. The method of embodiment 25, wherein homogenization of the liposome-drug suspension in step (c) is performed by microfluidization, sonication, extrusion, freeze-thaw, or a combination thereof
37. The method of embodiment 25, wherein the therapeutic agent does not contact the liposomal lipid prior to the formation of the liposomes.
38. The method of embodiment 25, wherein the liposomes are essentially unilamellar after step (c).
39. The method of embodiment 25, wherein the liposomes have a diameter of about 100 nm or less after step (c).
40. The method of embodiment 25, wherein the liposomes have a diameter of about 50 nm or less after step (c).
41. The method of embodiment 25, wherein the liposome suspension provided in step (a) comprises an additional therapeutic agent present in the aqueous medium and/or the liposomes.
42. The method of embodiment 25, wherein the composition of the aqueous medium inside and outside the liposomes is identical in steps (a), (b), and (c).
43. The method of embodiment 25, wherein the liposomal lipid comprises not more than 20% saturated fatty acids.
44. The method of embodiment 25, wherein the liposomal lipid comprises L-α-phosphatidylcholine.
45. The method of embodiment 25, wherein the method further comprises, after step (c), performing sterile filtration, lyophilization, or lyophilization and reconstitution with an aqueous medium.
46. The method of embodiment 25, wherein the therapeutic agent remains bound to the lipid bilayer phase of the liposomes after step (c) for at least 2 months upon storage in the aqueous medium at about 4° C.
47. The method of embodiment 25, wherein the therapeutic agent remains associated with the lipid bilayer phase after step (c) followed by lyophilization and storage for at least 1 year at ambient temperature and reconstitution in an aqueous medium.
48. The method of embodiment 25, wherein average liposome size after step (c) remains less than about 100 nm for at least 2 months upon storage in the aqueous medium at about 4° C.
49. The method of embodiment 25, wherein after step (c) followed by lyophilization, storage for at least 1 year at ambient temperature, and reconstitution in an aqueous medium, the average liposome size remains less than about 100 nm.
50. The method of embodiment 25, wherein said temperature is from about 15° C. to about 35° C.
51. The method of embodiment 25, wherein the lipid bilayer phase is liquid crystalline at said temperature.
52. The method of embodiment 25, wherein the lipid concentration of the liposome-drug suspension in steps (b) and (c) is from about 1 to about 7% by weight.
53. The method of embodiment 25, wherein the solid hydrophobic therapeutic agent added in step (b) provides a total concentration of the agent in the liposome-drug suspension of from about 1 to about 20 mg/mL.
54. The method of embodiment 25, further comprising:

(d) lyophilizing the homogenized liposome suspension, whereby said solvent is removed.

### Definitions

As used herein, and unless otherwise specified, “lipid” is understood to be a fatty acid, fatty acid salt, fatty alcohol, or phospholipid. Lipids may also be read to include sterols, including, but not limited to, cholesterol; sphingolipids, including, but not limited to, sphingomyelin; glycosphingolipids including, but not limited to, gangliosides, globocides and cerebrosides; and surfactant amines including, but not limited to, stearyl, oleyl and linoleyl amines.

As used herein, and unless otherwise specified, “phospholipid” is understood to be an amphiphilic derivative of glycerol, in which one of its hydroxyl groups is esterified with phosphoric acid and the other two hydroxyl groups are esterified with long-chain fatty acids that can be equal to or different from each other and can be saturated or unsaturated. A neutral phospholipid is generally one in which the other phosphoric acid hydroxyl is esterified by an alcohol substituted by a polar group (usually hydroxyl or amino) and whose net charge is zero. A phospholipid with a charge is generally one in which the other phosphoric acid hydroxyl is esterified by an alcohol substituted by a polar group and whose net charge is positive or negative.

Examples of phospholipids include, but are not limited to phosphatidic acid (“PA”), phosphatidylcholine (“PC”), phosphatidylglycerol (“PG”), phophatidylethanolamine (“PE”), phophatidylinositol (“PI”), and phosphatidylserine (“PS”), sphingomyelin (including brain sphingomyelin), lecithin, lysolecithin, lysophosphatidylethanolamine, cerebrosides, diarachidoylphosphatidylcholine (“DAPC”), didecanoyl-L-alpha-phosphatidylcholine (“DDPC”), dielaidoylphosphatidylcholine (“DEPC”), dilauroylphosphatidyl choline (“DLPC”), dilinoleoylphosphatidylcholine, dimyristoylphosphatidylcholine (“DMPC”), dioleoylphosphatidylcholine (“DOPC”), dipalmitoylphosphatidylcholine (“DPPC”), di stearoylphosphatidylcholine (“DSPC”), 1-palmitoyl-2-oleoyl-phosphatidylcholine (“POPC”), diarachidoylphosphatidylglycerol (“DAPG”), didecanoyl-L-alpha-phosphatidylglycerol (“DDPG”), dielaidoylphosphatidylglycerol (“DEPG”), dilauroylphosphatidylglycerol (“DLPG”), dilinoleoylphosphatidylglycerol, dimyristoylphosphatidylglycerol (“DWG”), diolcoylphosphatidylglycerol (“DOPG”), dipalmitoylphosphatidylglycerol (“DPPG”), distearoylphosphatidylglycerol (“DSPG”), 1-palmitoyl-2-oleoyl-phosphatidylglycerol (“POPG”), diarachidoylphosphatidylethanolaminc (“DAPE”), didecanoyl-L-alpha-phosphatidylethanolamine (“DDPE”), dielaidoylphosphatidylethanolamine (“DEPE”), dilauroylphosphatidylethanolamine (“DLPE”), dilinoleoylphosphatidylcthanol amine, dimyristoylphosphatidylethanolamine (“DMPE”), dioleoylphosphatidylethanolamine (“DOPE”), dipalmitoylphosphatidylethanolamine (“DPPE”), distearoylphosphatidylethanolamine (“DSPE”), 1-palmitoyl-2-oleoyl-phosphatidylethanolamine (“POPE”), diarachidoylphosphatidylinositol (“DAPI”), didecanoyl-L-alpha-phosphatidylinositol (“DDPI”), dielaidoylphosphatidylinositol (“DEPI”), dilauroylphosphatidylinositol (“DLPI”), dilinoleoylphosphatidylinositol, dimyristoylphosphatidylinositol (“DWI”), dioleoylphosphatidylinositol (“DOPI”), dipalmitoylphosphatidylinositol (“DPPI”), distearoylphosphatidylinositol (“DSPI”), 1-palmitoyl-2-oleoyl-phosphatidylinositol (“POPI”), diarachidoylphosphatidylserine (“DAPS”), didecanoyl-L-alpha-phosphatidylserine (“DDPS”), dielaidoylphosphatidylserine (“DEPS”), dilauroylphosphatidylserine (“DLPS”), dilinoleoylphosphatidylserine, dimyristoylphosphatidylserine (“DMPS”), dioleoylphosphatidylserine (“DOPS”), dipalmitoylphosphatidylserine (“DPPS”), distearoylphosphatidylserine (“DSPS”), 1-palmitoyl-2-oleoyl-phosphatidylserine (“POPS”), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, and 1-palmitoyl-2-oleoyl-sphingomyelin.

As used herein, an “antibody” can be a polyclonal or monoclonal antibody, and can be a naturally occurring or recombinant immunoglobulin molecule. An antibody can be any class of immunoglobulin, such as IgG, IgM, IgA, IgD or IgE. Antibodies for use in the invention include antibody analogs and derivatives, such as antibody fragments (Fab, Fc, Fv, and the like), diabodies, triabodies, minibodies, nanobodies, single-domain antibodies such as scFv, and antibody fusion proteins. They can be monospecific, bispecific or multispecific. Antibodies can be, for example, murine, chimeric, humanized, or human antibodies.

As used herein, and unless otherwise specified, “encapsulate” or “encapsulation” is understood to be the process of incorporating an active agent into liposomes or liposomal vesicles. The encapsulated active agent can remain in the aqueous interior or associate with membranes.

As used herein, and unless otherwise specified, the term “enhance” or “enhancing,” when used in connection with the solubility of a compound, means that the methods provided herein result in the increased solubility of the compound as compared to the solubility of the same compound in water. Specifically, the term “enhance” or “enhancing” means that, when the methods provided herein are used, the solubility of a compound increases about 20 percent or more, about 40 percent or more, about 60 percent or more, about 80 percent or more, about 100 percent or more, or about 200 percent or more of the solubility of the same compound in a reference solvent. In some embodiments, the reference solvent is water.

As used herein, and unless otherwise specified, the term “hydrophobic compound” or “hydrophobic therapeutic agent” means a compound with little or no water solubility. A hydrophobic compound or hydrophobic therapeutic agent can be an organic or inorganic molecule or a biomolecule of any size. In some embodiments, a hydrophobic compound has an intrinsic water solubility (i.e., water solubility of the unionized form) of less than about 20 percent by weight, about 15 percent by weight, about 10 percent by weight, about 5 percent by weight, about 1 percent by weight, about 0.1 percent by weight or about 0.01 percent by weight. In other embodiments, a hydrophobic compound has an intrinsic water solubility of less than about 10 mg/mL, about 7 mg/mL, about 5 mg/mL, about 3 mg/mL, about 1 mg/mL or about 0.1 mg/mL. In some embodiments, a hydrophobic compound or therapeutic agent can have, for example, an octanol-water partition coefficient (log P value) that is greater than about 0, greater than about 0.5, greater than about 1, greater than about 1.5, greater than about 2, greater than about 2.5, greater than about 3, greater than about 3.5, or greater than about 4. In some embodiments, the hydrophobic therapeutic agent is amphipathic. The hydrophobic therapeutic agent can interact with liposomal lipids via any type of non-covalent interaction, including electrostatic interactions, hydrogen bonding, and/or van der Waals interactions, in addition to hydrophobic interactions.

As used herein, or unless otherwise specified, the terms “aqueous medium” or “aqueous media” include any water based medium, e.g., water, saline solution, a sugar solution, a transfusion solution, a buffer, and any other readily available water-based medium. Further, an aqueous medium may contain one or more water soluble organic solvents. In the case of a parenteral solution, an aqueous medium is preferably sterile and suitable for use as a carrier of an active agent. Examples of aqueous media include, but are not limited to, water for injection, saline solution, Ringer's solution, D5W, or other solutions of water-miscible substances such as dextrose and other electrolytes.

As used herein, and unless otherwise specified, the term “fatty acid” means a compound whose structure is a carboxylic group attached to a hydrocarbon chain having one or more carbon atoms. The hydrocarbon chain may be saturated or unsaturated (i.e., alkyl, alkenyl or alkynyl hydrocarbon chains). Also, the hydrocarbon chain may be straight or branched. Moreover, in some embodiments, hydrogens in the hydrocarbon chain may be substituted.

As used herein, and unless otherwise specified, the term “fatty alcohol” means a compound whose structure is an alcohol group attached to a hydrocarbon chain having one or more carbon atoms. The hydrocarbon chain may be saturated or unsaturated (i.e., alkyl, alkenyl or alkynyl hydrocarbon chains). Also, the hydrocarbon chain may be straight or branched. Moreover, in some embodiments, hydrogens in the hydrocarbon chain may be substituted.

As used herein, and unless otherwise specified, the term “fatty acid salt” means a compound formed from a reaction between a fatty acid and an inorganic/organic base. In addition, the term encompasses a compound formed from a reaction between a fatty alcohol and an inorganic/organic acid. Examples of such acids include, but are not limited to, sulfuric and phosphoric acid. The hydrocarbon chain of the fatty acid salt may be saturated or unsaturated (i.e., alkyl, alkenyl or alkynyl hydrocarbon chains). In addition, the hydrocarbon chain may be straight or branched. Moreover, in some embodiments, hydrogens in the hydrocarbon chain may be substituted.

As used herein, and unless otherwise specified, the term “substituted” means a group substituted by one or more substituents including, but not limited to, alkyl, alkenyl, alkynyl, cycloalkyl, aroyl, halo, haloalkyl (e.g., trifluoromethyl), substituted or unsubstituted heterocycloalkyl, haloalkoxy (e.g., trifluoromethoxy), hydroxy, alkoxy, cycloalkyloxy, heterocylooxy, oxo, alkanoyl, aryl, substituted aryl, substituted or unsubstituted heteroaryl (e.g., indolyl, imidazolyl, furyl, thienyl, thiazolyl, pyrrolidyl, pyridyl, pyrimidyl and the like), arylalkyl, alkylaryl, heteroaryl, heteroarylalkyl, alkylheteroaryl, heterocyclo, aryloxy, alkanoyloxy, amino, alkylamino, aryl amino, aryl alkyl amino, cycloalkylamino, heterocycloamino, mono- and di-substituted amino, alkanoylamino, aroylamino, aralkanoylamino, substituted alkanoylamino, substituted arylamino, substituted aralkanoylamino, carbamyl (e.g., CONH.sub.2), substituted carbamyl (e.g., CONH-alkyl, CONH-aryl, CONH-arylalkyl or instances where there are two substituents on the nitrogen), carbonyl, alkoxycarbonyl, carboxy, cyano, ester, ether, guanidino, nitro, sulfonyl, alkylsulfonyl, aryl sulfonyl, aryl alkyl sulfonyl, sulfonamido (e.g., SO. sub.2NH.sub.2), substituted sulfonamido, thiol, alkylthio, arylthio, arylalkylthio, cycloalkylthio, heterocyclothio, alkylthiono, arylthiono and arylalkylthiono.

As used herein, and unless otherwise specified, the term “alkyl” means a saturated straight chain or branched non-cyclic hydrocarbon having 1-20 carbon atoms, preferably 1-10 carbon atoms and most preferably 1-4 carbon atoms. Representative saturated straight chain alkyls include -methyl, -ethyl, -n-propyl, -n-butyl, -n-pentyl, -n-hexyl, -n-heptyl, -n-octyl, -n-nonyl and -n-decyl; while saturated branched alkyls include -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methyl hexyl, 5-methylhexyl, 2,3-dimethyl butyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimethylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3 -ethylhexyl, 2-methyl-4-ethylhexyl, 2,2-diethylpentyl, 3,3 -diethylhexyl, 2,2-diethylhexyl, 3,3-diethylhexyl and the like. An alkyl group can be unsubstituted or substituted. Unsaturated alkyl groups include alkenyl groups and alkynyl groups, which are discussed below.

As used herein, and unless otherwise specified, the term “alkenyl” means a straight chain or branched non-cyclic hydrocarbon having 2-20 carbon atoms, preferably 2-10 carbon atoms, most preferably 2-6 carbon atoms, and including at least one carbon-carbon double bond.

Representative straight chain and branched (C₂-C₁₀) alkenyls include -vinyl, -allyl, -1-butenyl, -2-butenyl, -isobutylenyl, -1-pentenyl, -2-pentenyl, -3-methyl-l-butenyl, -2-methyl-2-butenyl, -2,3 -dimethyl -2-butenyl, -1-hexenyl, -2-hexenyl, -3 -hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like. The double bond of an alkenyl group can be unconjugated or conjugated to another unsaturated group. An alkenyl group can be unsubstituted or substituted.

As used herein, and unless otherwise specified, the term “alkynyl” means a straight chain or branched non-cyclic hydrocarbon having 2-20 carbon atoms, preferably 2-10 carbon atoms, most preferably 2-6 carbon atoms, and including at least one carbon-carbon triple bond. Representative straight chain and branched (C2-C10)alkynyls include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl, -1-heptynyl, -2-heptynyl, -6-heptynyl, -1-octynyl, -2-octynyl, -7-octynyl, -1-nonynyl, -2-nonynyl, -8-nonynyl, -1-decynyl, -2-decynyl, -9-decynyl, and the like. The triple bond of an alkynyl group can be unconjugated or conjugated to another unsaturated group. An alkynyl group can be unsubstituted or substituted.

As used herein, and unless otherwise specified, the term “pharmaceutically acceptable salt” refers to a salt prepared from pharmaceutically acceptable non-toxic acids or bases including inorganic acids and bases and organic acids and bases. Suitable pharmaceutically acceptable base addition salts for the compositions provided herein include, but are not limited to, metallic salts made from aluminum, calcium, lithium, magnesium, potassium, sodium, and zinc, or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art, see e.g., Remington's Pharmaceutical Sciences, 18^{th} ed., Mack Publishing, Easton Pa. (1990) or Remington: The Science and Practice of Pharmacy, 19th ed., Mack Publishing, Easton Pa. (1995).

As used herein, the term “hydrate” means a compound provided herein, or a salt thereof, that further includes a stoichiometric or non-stoichiometric amount of water bound by non-covalent intermolecular forces.

As used herein, the term “clathrate” means a compound provided herein, or a salt thereof in the form of a crystal lattice that contains spaces (e.g., channels) that have a guest molecule (e.g., a solvent or water) trapped within.

As used herein, and unless otherwise indicated, the term “prodrug” means a derivative of a compound that can hydrolyze, oxidize, or otherwise react under biological conditions (in vitro or in vivo) to provide an active compound. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Preferably, prodrugs of compounds with carboxyl functional groups are the lower alkyl esters of the carboxylic acid. Esterifying any of the carboxylic acid moieties present on the molecule conveniently forms the carboxylate esters. Prodrugs can typically be prepared using well-known methods, such as those described by Burger's Medicinal Chemistry and Drug Discovery 6th ed. (Donald J. Abraham ed., 2001, Wiley), and Design and Application of Prodrugs (H. Bundgaard ed., 1985, Harwood Academic Publishers Gmfh).

As used herein, and unless otherwise specified, the term “stable,” when used in connection with a formulation, means that the active agent of the formulation, when prepared using the methods provided herein, remains solubilized for a specified amount of time and does not significantly degrade or aggregate or become otherwise modified (e.g., as determined by HPLC). When referring to a suspension of liposomes, the term “stable” means that the liposomes, when prepared using the methods provided herein, remain dispersed in suspension for a specified amount of time and do not significantly degrade, aggregate, precipitate or become otherwise modified, such as by changing their size or number of lamellae. Stability of liposomes can be monitored by any known technique, such as by electron microscopy or dynamic light scattering).

As used herein, and unless otherwise specified, “temperature below the Tc” is understood to be a temperature which is lower than the Tc of the lipid having the lowest Tc, and “temperature greater than the Tc” is understood to be a temperature which is greater than the Tc of the lipid having the highest Tc.

As used herein, and unless otherwise specified, the term “harmful ingredient,” when used in connection with pharmaceutical compositions, means an ingredient commonly used in a pharmaceutical composition that may cause clinical side effects such as, but not limited to, hemolysis, hypersensitive reaction, peripheral neuropathies, and/or decrease in the bioavailability of the active ingredient of the composition. Examples of harmful ingredients include, but are not limited to: toxic solvents, including organic solvents such as ethanol, methanol, 1-propanol, 2-propanol, acetone, acetonitrile, ethyl acetate, methyl acetate, diethyl ether, dimethyl ether, diisopropyl ether, methyl tert-butyl ether (“MTBE”), tetrahydrofuran (“THF”), dichloromethane, chloroform, carbon tetrachloride, 1,2-dichloroethane, pentane, hexanes, heptane, petroleum ether, dioxane, ethylene glycol, diethylene glycol, diglyme, 1,2-dimethoxyethane, 1-butanol, 2-butanol, 2-butanone, benzene, toluene, dimethylsulfoxide (“DMSO”), dimethylformamide (“DMF”), hexamethylphosphoramide (“HMPA”), N-methylpyrrolidone, glycerin, nitromethane, triethyl amine, xylenes. CREMOPHOR™ EL, and polyethylene glycol (“PEG”); co-detergents or surfactants such as polysorbates (e.g., Tweens) or vitamin E; oils such as Castor oil or corn oil; proteins such as HSA; or any other biologic which is potential source of contamination.

As used herein, the term “water-miscible solvent” refers to a solvent that forms a single continuous phase when mixed with water at the ratio of solvent to water used. Miscibility can be determined by a variety of known methods, including visual inspection and other optical methods.

### BRIEF DESCRIPTION OF DRAWINGS

FIG. 1 depicts the percent body weight changes after injection of TAXOTERE and MIRADOCETAXEL (Q7DX3) to nude mice at different concentrations.

FIG. 2 depicts the percent body weight changes after injection of TAXOTERE and MIRADOCETAXEL (Q7DX3) to nude mice at 20 mg/kg dose.

FIG. 3 depicts the efficacy of TAXOTERE and MIRADOCETAXEL on human melanoma tumors.

FIG. 4 depicts the efficacy of TAXOTERE and MIRADOCETAXEL on human prostate tumors.

FIG. 5 depicts comparisons of docetaxel mean plasma concentrations in rats following a single 25 mg/kg dose of decetaxel in TAXOTERE and MIRADOCETAXEL formulations.

FIG. 6 is a schematic representation of different modes of encapsulation of one or more hydrophobic therapeutic agents by a liposome according to different embodiments of the invention.

FIG. 7A shows a control elution profile for size exclusion HPLC of adalimumab antibody without added liposomes. FIG. 7B shows an elution profile for size exclusion HPLC of liposome-encapsulated trastuzumab antibody. FIG. 7C shows an elution profile for size exclusion HPLC of liposome-encapsulated adalimumab antibody.

FIG. 8 shows particle sizes of MIRADOCETAXEL-containing liposomes after lyophilization and storage at −20° C. for 2 years.

### DETAILED DESCRIPTION

Provided herein are methods for the preparation of liposomes containing a hydrophobic therapeutic agent. The methods allow the hydrophobic therapeutic agent to be effectively solubilized in aqueous media at high concentrations by incorporating it into liposomes. The liposomes are stable, and their production reduces or avoids the use of organic solvents, surfactants, and other harmful ingredients.

In some embodiments, two or more different hydrophobic therapeutic agents are incorporated into liposomes by any method of the invention. In some embodiments, the hydrophobic therapeutic agent is encapsulated into liposomes as a solid, without presenting an organic solvent solution of the agent to the liposomes. In other embodiments, the hydrophobic therapeutic agent is contacted with the preformed liposomes dissolved in a liquid medium including or consisting of solvent.

The hydrophobic therapeutic agent can interact with liposomal lipids via any type of non-covalent interaction, including hydrophobic interactions, electrostatic (ionic) interactions, hydrogen bonding, and/or van der Waals interactions. Preferably, hydrophobic interactions represent a significant component of the binding interaction between the hydrophobic agent molecule and the liposomal lipid molecules. Preferably the binding interaction is entirely non-covalent, and preferably the hydrophobic compound and lipid molecules have not been chemically modified to create a covalent or non-covalent association between the two. A hydrophobic therapeutic agent can be incorporated into or attached to liposomes in a variety of ways. The hydrophobic therapeutic agent can be partially or completely encapsulated by the liposomal lipid phase. In some embodiments, the hydrophobic therapeutic agent is reversibly attached to the liposome lipid bilayer. In some embodiments, the hydrophobic therapeutic agent is located all or in part in the hydrophobic interior or core of the lipid bilayer, associated with the acyl chains. In some embodiments, the hydrophobic therapeutic agent is located all or in part at the outer and/or inner surface of the lipid bilayer, and interacts with the polar groups of the liposome lipids. In some embodiments, the hydrophobic therapeutic agent is bound to the liposomes by non-covalent interactions with both the polar and non-polar groups of the liposome lipids.

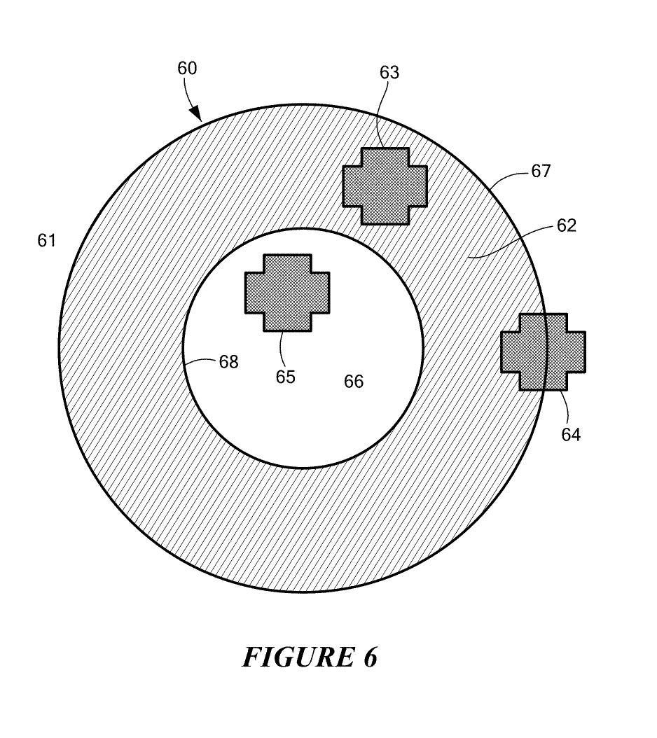

FIG. 6 shows a schematic representation of liposome 60 suspended in an aqueous medium 61 containing an encapsulated hydrophobic therapeutic agent. In one embodiment, hydrophobic therapeutic agent 63 preferentially associates with and binds to the hydrophobic core of the lipid bilayer 62. In another embodiment, hydrophobic therapeutic agent 64 associates with and binds to both the hydrophobic interior of lipid bilayer 62 and outer 67 and/or inner 68 surfaces of the lipid bilayer, being only partially located within the hydrophobic core of the lipid bilayer. In yet another embodiment, hydrophobic therapeutic agent 65 can be located within aqueous interior 66 of the liposome. In some embodiments, the hydrophobic therapeutic agent is found at two or more of locations 62, 67, 68, and 66.

In one embodiment, a hydrophobic therapeutic agent is added to and incorporated within liposomal membranes from a solid form. The present inventors have discovered that a hydrophobic compound can be incorporated into liposomes by exposing the hydrophobic compound, provided in a solid form, such as granules, crystals, or a precipitate, to pre-formed liposomal membranes suspended in an aqueous medium. The present inventors discovered that liposomal membranes are capable of solubilizing hydrophobic or amphipathic compounds, such as pharmaceutical agents, in a manner analogous to the uptake of such compounds into micelles, even though the exterior surface of the bilayer contains charged and hydrophilic moieties. The method includes the steps of: (a) providing (i) a liposome suspension containing a plurality of preformed liposomes suspended in an aqueous medium, the liposomes containing lipid forming a lipid bilayer phase, and (ii) a solid form of a hydrophobic therapeutic agent; (b) adding the solid form of the hydrophobic therapeutic agent to the liposome suspension, thereby forming a liposome-drug suspension; and (c) homogenizing the liposome-drug suspension. The hydrophobic therapeutic agent is incorporated into the lipid bilayer phase. Step (c) is performed without the addition of heat, such as at a temperature at or below ambient temperature. In some embodiments, steps (b) and/or (c) are performed without the use of solvent. In some embodiments, steps (b) and/or (c) are performed without the use of surfactant.

Another aspect of the invention is a method of incorporating a hydrophobic therapeutic agent from a solvent-containing solution into preformed liposomes: The method includes the steps of: (a) providing (i) a liposome suspension containing a plurality of preformed liposomes suspended in an aqueous medium, the liposomes containing lipid forming a lipid bilayer phase, and (ii) a therapeutic agent concentrate comprising a hydrophobic therapeutic agent dissolved in a liquid medium comprising or consisting of solvent; (b) adding the therapeutic agent concentrate to the liposome suspension to form a liposome-drug suspension, wherein the total concentration of solvent in the liposome-drug suspension is not more than 10 weight percent; and (c) homogenizing the liposome-drug suspension. The hydrophobic therapeutic agent is incorporated into the lipid bilayer phase. Step (c) can be performed without the addition of heat, such as at a temperature at or below ambient temperature. The liquid medium of the therapeutic agent concentrate can have any composition that is capable of completely dissolving the hydrophobic therapeutic agent. In some embodiments, the liquid medium consists of a solvent or a mixture of two or more solvents. In some embodiments, the liquid medium consists of or includes an organic solvent. In some embodiments, the liquid medium consists of a water-miscible solvent. In some embodiments, the liquid medium consists of 100% ethanol. In some embodiments, the liquid medium contains water in addition to a water-miscible solvent. In some embodiments, the liquid medium includes a solvent or a mixture of two or more solvents.

In embodiments that contain a water-miscible solvent, the water-miscible solvent can be selected from the group consisting of alcohols, ketones, ethers, organic acids, organic bases, and mixtures thereof. In some embodiments, the water-miscible solvent is selected from the group consisting of ethanol, propanol, isopropanol, butanol, isobutanol, and dimethyl sulfoxide (DMSO). In some embodiments, the water-miscible solvent is ethanol. In some embodiments, the water-miscible solvent is ethanol and the liquid medium contains at least 50% ethanol, at least 60% ethanol, at least 70% ethanol, at least 80% ethanol, at least 90% ethanol, at least 95% ethanol, or at least 98% ethanol, with the remainder being preferably water.

In some embodiments, the total concentration of solvent in the liposome-drug suspension in step (b) is at most 7 weight percent, at most 5 weight percent, at most 4 weight percent, at most 3 weight percent, at most 2 weight percent, or at most 1 weight percent.

In some embodiments, steps (b) and/or (c) are performed using a limited concentration of surfactant, such as at most 1 weight percent, at m 0.5 weight percent, at most 0.25 weight percent, at most 0.1 weight percent, or at most 0.05 weight percent. In some embodiments, the molar ratio of liposomal lipid to surfactant is at least 100:1, at least 200:1, at least 300:1, at least 500:1, or at least 1000:1. In some embodiments, step (c) is performed without the use of surfactant. Without intending to limit the invention to any particular mechanism, the surfactant can enhance uptake of a hydrophobic compound into liposomal membranes by forming micelles containing the compound, which then partitions into the liposomal membranes, or by slightly disrupting the lipid bilayer structure so as to promote uptake of the compound.

In some embodiments, the method of incorporation of a hydrophobic compound into liposomal membranes is performed without adding heat, such as by performing the method at ambient temperature. Ambient temperature can be a temperature is from about 15° C. to about 35° C. In some embodiments, the method is conducted at a temperature in which the lipid bilayer phase is liquid crystalline.

In some embodiments, the hydrophobic therapeutic agent is a small molecule, such as a molecule having a molecular weight of 1500 daltons or less. Examples of small molecules include, but are not limited to, lapachone (β-lapachone), taxanes (including, but not limited to, taxol, 7-epitaxol, 7-acetyl taxol, 10-desacetyltaxol, 10-desacetyl-7-epitaxol, 7-xylosyltaxol, 10-desacetyl-7-sylosyltaxol, 7-glutaryltaxol, 7-N,N-dimethylglycycltaxol, 7-L-alanyltaxol, TAXOTERE, and mixtures thereof), paclitaxel, colchicine, transferrin, cyclosporines, cyclosporin A, ketoprofen, propofol, acetylsalicylic acid, acetaminophen, amphotericin, digoxin, doxorubicin, daunorubicin, epirubicin, idarubicin, angiogenesis inhibitors (e.g, vitaxin, carboxyamidotriazole, combretastatin A-4, fumagillin analogs (e.g., TNP-470), CM101, IFN-alpha, interleukin-10, interleukin-12, platelet factor-4, suramin, SU5416, thrombospondin, VEGFR antagonists, angiostatin, endostatin, 2-methoxyestradiol, tecogalan, thalidomide, prolactin, linomide, angiopoietin-1, basic fibroblast growth factor, vascular endothelial growth factor), vinca-alkaloids (e.g., vinblastine, vincristine, vindesin, etoposide, etoposide phosphate, and teniposide), cytarabine, actinomycin, etoposide, bleomycin, gentamycin, cyclophosphamide, methotrexate, streptozotocin, cytosine, beta-D-arabinofuranoside-5′-triphosphate, cytochrome C, cisplatin, N-phosphono-acetyl-L-aspartic acid, 5-fluoroorotic acid, acyclovir, zidovudine, interferons, aminoglycosides, cephalosporins, tetracyclines, propranolol, timolol, labetolol, clonidine, hydralazine, imipramine, amitriptyline, doxepim, phenyloin, diphenhydramine, chlorpheniramine, promethazine, prostaglandins, methotrexate, progesterone, testosterone, estradiol, estrogen, epirubicin, beclomethasone and esters, vitamin E, cortisone, dexamethasone and esters, betamethasone valerate, biphenyl dimethyl dicarboxylic acid, calcitonins, camptothecin, captopril, cephazoline, chloroquinine, chlorothiazole, co-agulation factors VIII and IX, d-alpha-tocopherol, dexamethasone, dichlofenac, etoposide, feldene, flubiprofen, 5-fluorouracil, fluoxetine, fusidic acid, gentamicin, glyburide, granisetron, growth hormones, indomethacin, insulin, itraconazole, ketoconazole, methotrexate, metronidazole, minoxidil, mitomycin, nafcillin, naproxen, ondansetron, oxyphenbutazone, parazosin, physostigmine, piroxicam, prednisolone, primaquine, quinine, ramipril, taxotane, tenoxicam, terazosin, triamcinol one, urokinase, opioid analgesics (e.g., alfentanil, anileridine, codiene, diamorphine, fentanyl, hydrocodone, hydromorphone, meperidine, morphine, oxycodone, oxymorphone, propoxyphene, sufentanil, pentazocine and nalbuphine), non-steroidal anti-inflammatory drugs (e.g., aspirin, indometacin, ibuprofen, mefenamic acid and phenylbutazone), angiotensin converting enzyme (“ACE”) inhibitors (e.g., captoprilpolyene), protein kinase C inhibitors, antibiotics (e.g., imidazole and triazole antibiotics), folic acid, anthracycline antibiotics, tricathecums, microbial ribosomal-inactivating toxins (e.g., gelonin, abrin, ricin A chain, Pseudomonas exotoxin, diptheria toxin, pokeweed antiviral peptide), pipecolic acid derivatives (e.g., tacrolimus), plant alkaloids, dyes, radioisotope-labeled compounds, radiopaque compounds, radiosensitizers (e.g., 5-chloro-2′-deoxyuridine, 5-bromo-2′-deoxyuridine and 5-iodo-2′-deoxyuridine), fluorescent compounds, mydriatic compounds, bronchodilators, local anesthetics (e.g., dibucaine and chlorpromazine), antifungal agents (e.g., miconazole, terconazole, econazole, isoconazole, butaconazole, clotrimazole, itraconazole, nystatin, naftifine and amphotericin B), antiparasitic agents, hormones, hormone antagonists, immunomodulators, neurotransmitter antagonists, anti-diabetic agents, antiglaucoma agents, vitamins, narcotics, and imaging agents. For additional disclosure of therapeutic small molecules, see Gilman et al., Goodman and Gilman's: The Pharmacological Basis of Therapeutics, 10th ed., McGraw-Hill, New York, 2001; The Merck Manual of Diagnosis and Therapy, Berkow, M. D. et al. (eds.), 17th Ed., Merck Sharp & Dohme Research Laboratories. Rahway, N.J., 1999; Cecil Textbook of Medicine, 20th Ed., Bennett and Plum (eds.), W. B. Saunders, Philadelphia, 1996.

In some embodiments, the hydrophobic therapeutic agent is an antibody or antigen-binding fragment thereof. Examples of antibodies include, but are not limited to, adalimumab, abciximab, alefacept, alemtuzumab, basiliximab, belimumab, bezlotoxumab, canakinumab, certolizumab pegol, cetuximab, daclizumab, denosumab, efalizumab, golimumab, inflectra, ipilimumab, ixekizumab, natalizumab, nivolumab, olaratumab, omalizumab, palivizumab, panitumumab, pembrolizumab, rituximab, tocilizumab, trastuzumab, secukinumab, ustekinumab. In some embodiments, the hydrophobic therapeutic agent is a protein, such as a recombinant protein.

After incorporation of the hydrophobic compound into liposomes, it is desirable to homogenize the liposomes to produce a uniform and stable population of liposomes containing the compound. Any suitable method known in the art can be used for homogenization of the suspension. In some embodiments, homogenization is performed with a MICROFLUIDIZER or a high-pressure homogenizer, by sonication or ultrasonication of the suspension, by extrusion or microextrusion of the suspension through a filter or similar structure, by employing freeze/thaw cycles, or any combination thereof. Homogenization typically results in a homogenous population of small unilamellar liposomes. In some embodiments, the liposomes are essentially unilamellar after step (c). In some embodiments, after step (c) the liposomes have a diameter of about 100 nm or less, about 90 nm or less, about 80 nm or less, about 70 nm or less, about 50 nm or less, about 40 nm or less, about 30 nm or less, about 20 nm or less, or about 10 nm or less. In some embodiments, the homogenization method can be chosen to provide high efficiency incorporation of the therapeutic agent. In some embodiments, at least about 80% of the hydrophobic therapeutic agent is associated with the lipid bilayer phase after step (c), or at least about 85%, at least about 90%, at least about 95%, at least about 98%, at least about 99%, or at least about 100%.

In certain embodiments, the resulting liposomes are less than about 1 μm in diameter.

In one embodiment, the resulting liposomes are less than about 500 nm in diameter.

In one embodiment, the resulting liposomes are less than about 100 nm in diameter.

In one embodiment, at least one of the lipids is a phospholipid or a mixture of phospholipids. Examples of phospholipids include, but are not limited to, phosphatidic acid (“PA”), phosphatidylcholine (“PC”), phosphatidylglycerol (“PG”), phophatidylethanolamine (“PE”), phophatidylinositol (“PI”), and phosphatidylserine (“PS”), sphingomyelin (including brain sphingomyelin), lecithin, lysolecithin, lysophosphatidylethanolamine, cerebrosides, diarachidoylphosphatidylcholine (“DAPC”), didecanoyl-L-alpha-phosphatidylcholine (“DDPC”), dielaidoylphosphatidylcholine (“DEPC”), dilauroylphosphatidylcholine (“DLPC”), dilinoleoylphosphatidylcholine, dimyristoylphosphatidylcholine (“DMPC”), dioleoylphosphatidylcholine (“DOPC”), dipalmitoylphosphatidylcholine (“DPPC”), di stearoylphosphatidylcholine (“DSPC”), 1-palmitoyl-2-oleoyl-phosphatidylcholine (“POPC”), diarachidoylphosphatidylglycerol (“DAPG”), didecanoyl-L-alpha-phosphatidylglycerol (“DDPG”), dielaidoylphosphatidylglycerol (“DEPG”), dilauroylphosphatidylglycerol (“DLPG”), dilinoleoylphosphatidylglycerol, dimyristoylphosphatidylglycerol (“DWG”), dioleoylphosphatidylglycerol (“DOPG”), dipalmitoylphosphatidylglycerol (“DPPG”), di stearoylphosphatidylglycerol (“DSPG”), 1-palmitoyl-2-oleoyl-phosphatidylglycerol (“POPG”), diarachidoylphosphatidylethanolamine (“DAPE”), didecanoyl-L-alpha-phosphatidylethanolamine (“DDPE”), dielaidoylphosphatidylethanolamine (“DEPE”), dilauroylphosphatidylethanolamine (“DLPE”), dilinoleoylphosphatidylethanolamine, dimyristoylphosphatidylethanolamine (“DMPE”), dioleoylphosphatidylethanolamine (“DOPE”), dipalmitoylphosphatidylethanolamine (“DPPE”), distearoylphosphatidylethanolamine (“DSPE”), 1-palmitoyl-2-oleoyl-phosphatidylethanolamine (“POPE”), diarachidoylphosphatidylinositol (“DAPI”), didecanoyl-L-alpha-phosphatidylinositol (“DDPI”), dielaidoylphosphatidylinositol (“DEPT”), dilauroylphosphatidylinositol (“DLPI”), dilinoleoylphosphatidylinositol, dimyristoylphosphatidylinositol (“DWI”), dioleoylphosphatidylinositol (“DOPI”), dipalmitoylphosphatidylinositol (“DPPI”), distearoylphosphatidylinositol (“DSPI”), 1-palmitoyl-2-oleoyl-phosphatidylinositol (“POPI”), diarachidoylphosphatidylserine (“DAPS”), didecanoyl-L-alpha-phosphatidylserine (“DDPS”), dielaidoylphosphatidylserine (“DEPS”), dilauroylphosphatidylserine (“DLPS”), dilinoleoylphosphatidylserine, dimyristoylphosphatidylserine (“DMPS”), dioleoylphosphatidylserine (“DOPS”), dipalmitoylphosphatidyl serine (“DPPS”), distearoylphosphatidylserine (“DSPS”), 1-palmitoyl-2-oleoyl-phosphatidylserine (“POPS”), diarachidoyl sphingomyelin, didecanoyl sphingomyelin, dielaidoyl sphingomyelin, dilauroyl sphingomyelin, dilinoleoyl sphingomyelin, dimyristoyl sphingomyelin, sphingomyelin, dioleoyl sphingomyelin, dipalmitoyl sphingomyelin, distearoyl sphingomyelin, and 1-palmitoyl-2-oleoyl-sphingomyelin. In some embodiments, the phospholipid is soy phosphatidylcholine.

The phospholipids provided herein may be chiral or achiral. The chiral phospholipids provided herein may be D- or L-phospholipids, for example, L-α-phosphatidylcholine or L-3-phosphatidylcholine.

In some embodiments, the liposomes include unsaturated lipids. In some embodiments, saturated fatty acids constitute at most 20% of total lipids in the liposomes, or at most 15%, at most 10%, at most 5%, or at most 1% of total lipids.

In some embodiments, the lipid concentration of the liposome-drug suspension is about 1% by weight, 3% by weight, 5% by weight, or 7% by weight, before and/or after homogenization.

In one embodiment, L-α-phosphatidylcholine is used in the methods provided herein.

In another embodiment, provided herein is a method for the preparation of liposomes, the method comprising:

(a) combining sodium oleate and L-α-phosphatidylcholine in an aqueous medium at ambient temperature;

(b) dispersing sodium oleate and L-α-phosphatidylcholine in the aqueous medium; and

(c) adding one or more sugars to the resulting mixture, thereby forming a solution of liposomes.

In another embodiment, the resulting solution contains 10% by weight trehalose.

In one embodiment, the hydrophobic therapeutic agent is a pharmaceutically acceptable salt, hydrate, clathrate or prodrug of an active pharmaceutical compound.

Examples of sugars that may be used in the methods provided herein include, but are not limited to, sucrose, glucose, fructose, lactose, maltose, mannose, galactose and trehalose. In one embodiment, the sugar is trehalose.

In one embodiment, the liposomal preparation is suitable for parenteral administration to a patient suffering from one or more diseases or disorders. In one embodiment, the patient is a human.

In one embodiment, the aqueous medium contains one or more additional active agents, or pharmaceutically acceptable salts, hydrates, clathrates or prodrugs thereof. Examples of active agents include, but are not limited to, In one embodiment, the additional active agent is a hydrophobic compound, or a compound with poor solubility in water.

As provided herein, the sequence of the addition of the hydrophobic active agent(s) results in enhanced solubility of the active agent(s). The conventional method of incorporating a hydrophobic drug to liposome is by adding the drug to lipid before liposome preparation. See, e.g., Immordino, M. L. et al., Journal of Controlled Release, 2003, 91: 417-429, which shows that, by the conventional process, liposome incorporation of docetaxel, a hydrophobic antitumor agent is only 0.3 to 0.7 mg/mL. In contrast the present method increases incorporation of docetaxel to 5 mg/mL, a 10-fold increase.

The present methods include addition of the hydrophobic therapeutic agent(s) after formation of the liposomes, thereby resulting in enhanced solubility of the hydrophobic therapeutic agent(s) compared to other methods of loading hydrophobic therapeutic agents into liposomes. In certain embodiments, the therapeutic agent does not contact the liposomal lipid prior to the formation of the liposomes. In one embodiment, the solubility of the hydrophobic therapeutic agent(s) in liposomes is increased by at least about two-fold, three-fold, five-fold, or ten-fold compared to the conventional process. In some embodiments, the amount of solid hydrophobic therapeutic agent added in step (b) is from about 1 to about 20 mg/mL, such as about 3 mg/mL, about 5 mg/mL, about 7 mg/mL, about 10 mg/mL, about 12 mg/mL, about 15 mg/mL, about 17 mg/mL, or about 19 mg/mL. In some embodiments, the molar ratio of therapeutic agent to lipid is at least about 1:10, such as at least about 1:9, at least about 1:8, at least about 1:7, at least about 1:6, or at least about 1:5.

In certain embodiments, the sequence of the addition of hydrophobic therapeutic agent results in greater efficiency of incorporation of the hydrophobic therapeutic agent into liposome. In certain embodiments, the efficiency of incorporation is at least 50%, 60%, 70%, 80%, 90%, 95%, 98%, 99% or 100%. In certain embodiments, the efficiency of incorporation is about 90%, 95%, 98%, 99% or 100%.

Without being limited to a particular theory or mechanism, the increase in incorporation of hydrophobic therapeutic agent(s) into pre-made liposomes (e.g., to a concentration of about 5 mg/mL) may be due to an increase in the surface area of the liposomes. In some embodiments, the liposome suspension provided in step (a) includes an additional active agent present in the aqueous medium and/or the liposomes. In one embodiment, the additional active agent is transferrin, or a pharmaceutically acceptable salt, hydrate, clathrate or prodrug thereof In one embodiment, the additional active agent is cyclosporine, or a pharmaceutically acceptable salt, hydrate, clathrate or prodrug thereof. In one embodiment, the additional active agent is lapachone, or pharmaceutically acceptable salts, hydrates, clathrates or prodrugs thereof In one embodiment, the additional active agents are transferrin and lapachone, or pharmaceutically acceptable salts, hydrates, clathrates or prodrugs thereof.

In certain embodiments, the methods provided herein result in stable solutions, compositions or formulations comprising liposomes and hydrophobic therapeutic agent(s). Without being bound by any theory, it is believed that the reduced concentration or complete absence of solvent during the preparation of the liposomes generates liposome suspensions with little or no residual solvent. In the absence of solvent, liposomes are more stable, allowing the therapeutic agent to remain associated with the lipid bilayer phase of the liposomes for longer, and preventing liposome aggregation. In some embodiments, the stable solutions comprise one or more additional active agents.

In some embodiments, the liposomes containing a hydrophobic therapeutic agent remain dispersed in solution for a specified amount of time and do not significantly degrade, aggregate, precipitate or become otherwise modified (e.g., as determined by dynamic light scattering).

In some embodiments, the hydrophobic therapeutic agent(s) remains solubilized for a specified amount of time and does not significantly degrade, aggregate or become otherwise modified (e.g., as determined by HPLC).

In some embodiments, about 70 percent or greater, about 80 percent or greater or about 90 percent or greater of the one or more hydrophobic therapeutic agents remain solubilized after a week after dilution with an acceptable diluent at an elevated temperature (e.g., about 35° C. or higher).

In other embodiments, about 70 percent or greater, about 80 percent or greater or about 90 percent or greater of the one or more hydrophobic therapeutic agents remain solubilized after a week after dilution with an acceptable diluent at room temperature (e.g., from about 15° C. to about 35° C.).

In other embodiments, about 70 percent or greater, about 80 percent or greater or about 90 percent or greater of the one or more hydrophobic therapeutic agents remains solubilized after a week at a reduced temperature (e.g., about 10° C. or lower, or about 4° C. or lower).

In some embodiments, at least about 70%, at least about 80% or at least about 90% of the hydrophobic therapeutic agent remains associated with the lipid bilayer phase of the liposomes for at least 2 months, at least 3 months, at least 4 months, at least 5 months, or at least 6 months upon storage in the aqueous medium at a reduced temperature (e.g., about 10° C. or lower, or about 4° C. or lower).

In some embodiments, average liposome size after step (c) remains about 100 nm or less, about 90 nm or less, about 80 nm or less, about 70 nm or less, about 50 nm or less, about 40 nm or less, about 30 nm or less, about 20 nm or less, or about 10 nm or less for at least 2 months upon storage in the aqueous medium at at a reduced temperature (e.g., about 10° C. or lower, or about 4° C. or lower).

In some embodiments, the method further includes, after step (c), performing sterile filtration, lyophilization, or lyophilization and reconstitution with an aqueous medium. In one embodiment, the lyophilized formulation is reconstituted in aqueous solution at desirable higher or lower concentrations. In some embodiments, the therapeutic agent remains associated with the lipid bilayer phase after lyophilization and storage at ambient temperature for at least 1 year, at least 2 years or at least 3 years followed by reconstitution in an aqueous medium. In some embodiments, the reconstituted liposomes have an average size of about 100 nm or less, about 90 nm or less, about 80 nm or less, about 70 nm or less, about 50 nm or less, about 40 nm or less, about 30 nm or less, about 20 nm or less, or about 10 nm or less.

In certain embodiments, the methods provided herein result in enhanced solubility of the hydrophobic therapeutic agent(s), as compared to the solubility of the same hydrophobic therapeutic agent in an aqueous medium. Specifically, when the methods provided herein are used, the solubility of the hydrophobic therapeutic agent increases about 20 percent or more, about 40 percent or more, about 60 percent or more, about 80 percent or more, about 100 percent or more, or about 200 percent or more of the solubility of the same hydrophobic therapeutic agent(s) in a reference solvent. In some embodiments, the reference solvent is water.

The methods disclosed herein do not require the establishment of gradients within the aqueous medium, such as osmotic gradients or pH gradients, in order to achieve high levels of drug loading into the liposomes. Therefore, the composition of the aqueous medium internal and external to the liposomes can be identical in all aspects, including the presence and concentration of ions, salts, sugars and other excipients; presence and concentration of active agents; and pH and osmolality levels. In certain embodiments, the composition of the aqueous medium inside and outside the liposomes is identical in steps (a), (b), and (c).

In one embodiment, the liposomal preparation is suitable for parenteral administration to a patient suffering from one or more diseases or disorders. In some embodiments, the disease is cancer. In one embodiment, the patient is a human.

Also provided herein is a method of treating a disease or disorder using a liposomal composition provided herein. In some embodiments, the disease or disorder includes, but is not limited to, oncological disorders, proliferative disorders, central nervous system disorders, autoimmune disorders, and inflammatory diseases or disorders.

Proliferative disorders (e.g. cancer) that may be treated by the methods provided herein include, but are not limited to, neoplasms, tumors (malignant and benign) and metastases, or any disease or disorder characterized by uncontrolled cell growth. The cancer may be a primary or metastatic cancer. Specific examples of cancers that can be prevented, managed, treated or ameliorated in accordance with the methods of the invention include, but are not limited to, cancer of the head, neck, eye, mouth, throat, esophagus, chest, bone, lung, colon, rectum, stomach, prostate, breast, ovaries, kidney, liver, pancreas, and brain. Additional cancers include, but are not limited to, the following: leukemias (e.g., acute leukemia, acute lymphocytic leukemia), acute myelocytic leukemias (e.g., myeloblastic, promyelocytic, myelomonocytic, monocytic, erythroleukemia leukemias and myelodysplastic syndrome), chronic leukemias (e.g., chronic myelocytic (granulocytic) leukemia, chronic lymphocytic leukemia, hairy cell leukemia), polycythemia vera, lymphomas (e.g., Hodgkin's disease, non-Hodgkin's disease), multiple myelomas (e.g., smoldering multiple myeloma, nonsecretory myeloma, osteosclerotic myeloma, plasma cell leukemia, solitary plasmacytoma and extramedullary plasmacytoma), Waldenstrom's macroglobulinemia, monoclonal gammopathy of undetermined significance, benign monoclonal gammopathy, heavy chain disease, bone and connective tissue sarcomas (e.g., bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma), brain tumors (e.g., glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma), breast cancer (e.g., adenocarcinoma, lobular (small cell) carcinoma, intraductal carcinoma, medullary breast cancer, mucinous breast cancer, tubular breast cancer, papillary breast cancer, Paget's disease, and inflammatory breast cancer), adrenal cancer (e.g., pheochromocytom and adrenocortical carcinoma), thyroid cancer (e.g., papillary or follicular thyroid cancer, medullary thyroid cancer and anaplastic thyroid cancer), pancreatic cancer (e.g., insulinoma, gastrinoma, glucagonoma, vipoma, somatostatin-secreting tumor, and carcinoid or islet cell tumor), pituitary cancers (e.g., Cushing's disease, prolactin-secreting tumor, acromegaly, and diabetes insipius), eye cancers (e.g., ocular melanoma such as iris melanoma, choroidal melanoma, and cilliary body melanoma, and retinoblastoma), vaginal cancers (e.g., squamous cell carcinoma, adenocarcinoma, and melanoma), vulvar cancer (e.g., squamous cell carcinoma, melanoma, adenocarcinoma, basal cell carcinoma, sarcoma, and Paget's disease), cervical cancers (e.g., squamous cell carcinoma, and adenocarcinoma), uterine cancers (e.g., endometrial carcinoma and uterine sarcoma), ovarian cancers (e.g., ovarian epithelial carcinoma, borderline tumor, germ cell tumor, and stromal tumor), esophageal cancers (e.g., squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma), stomach cancers (e.g., adenocarcinoma, fungating (polypoid), ulcerating, superficial spreading, diffusely spreading, malignant lymphoma, liposarcoma, fibrosarcoma, and carcinosarcoma), colon cancers, rectal cancers, liver cancers (e.g., hepatocellular carcinoma and hepatoblastoma, gallbladder cancers such as adenocarcinoma), cholangiocarcinomas (e.g., pappillary, nodular, and diffuse), lung cancers (e.g., non-small cell lung cancer, squamous cell carcinoma (epidermoid carcinoma), adenocarcinoma, large-cell carcinoma and small-cell lung cancer), testicular cancers (e.g., germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, adenocarcinoma, leiomyosarcoma, and rhabdomyosarcoma), penile cancers, oral cancers (e.g., squamous cell carcinoma), basal cancers, salivary gland cancers (e.g., adenocarcinoma, mucoepidermoid carcinoma, and adenoidcystic carcinoma), pharynx cancers (e.g., squamous cell cancer, and verrucous), skin cancers (e.g., basal cell carcinoma, squamous cell carcinoma and melanoma, superficial spreading melanoma, nodular melanoma, lentigo malignant melanoma, acral lentiginous melanoma), kidney cancers (e.g., renal cell cancer, adenocarcinoma, hypernephroma, fibrosarcoma, transitional cell cancer (renal pelvis and/or uterer)), Wilms' tumor, bladder cancers (e.g., transitional cell carcinoma, squamous cell cancer, adenocarcinoma, carcinosarcoma), myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas, follicular lymphomas, carcinomas with p53 mutations, hormone dependent tumors of the breast, prostate and ovary, precancerous lesions such as familial adenomatous polyposis, and myelodysplastic syndromes.

Other specific diseases and disorders that may be treated by the methods provided herein include, but are not limited to, the following: allergic disorders, inflammation, asthma, arthritis, encephalitis, rheumatoid arthritis, osteoarthritis, psoriatic arthritis, inflammatory osteolysis, chronic or acute obstructive pulmonary disease, chronic or acute pulmonary inflammatory disease, inflammatory bowel disease, Crohn's Disease, gout, Bechet's Disease, Henoch-Schonlein purpura (“HSP”), septic shock, sepsis, meningitis, colitis, inflammation due to reperfusion, psoriasis, fibrosis including pulmonary fibrosis, Parkinson's disease, bradykinesi a, muscle rigidity, Parkinsonian tremor, Parkinsonian gait, motion freezing, depression; defective long-term memory, Rubinstein-Taybi syndrome (RTS), dementia, sleep disorders, insomnia, postural instability, hypokinetic disorders, hyperkinetic disorders, synuclein disorders, multiple system atrophies, striatonigral degeneration, olivopontocerebellar atrophy, Shy-Drager syndrome, motor neuron disease with parkinsonian features, Lewy body dementia, Tau pathology disorders, progressive supranculear palsy, corticobasal degeneration, frontotemporal dementia; amyloid pathology disorders, mild cognitive impairment, Alzheimer disease, Alzheimer disease with parkinsonism, Wilson disease, Hallervorden-Spatz disease, Chediak-Hagashi disease, SCA-3 spinocerebellar ataxia, X-linked dystonia parkinsonism, Huntington disease, prion disease, chorea, ballismus, dystonia tremors, Amyotrophic Lateral Sclerosis (“ALS”), CNS trauma, myoclonus, and diseases or disorders associated with undesired immune reaction (e g, organ rejection associated with an organ transplant).

### EXAMPLES

### Example 1

### Preparation of 6% L-α-Phosphatidylcholine (Soy) Liposome

6 g of L-α-Phosphatidylcholine (Soy) was dispersed in 100 mL of water using a magnetic stirrer at 200 rpm for 10 minutes at ambient temperature. The dispersed liposome (multilayer) was passed through a Microfluidic homogenizer at 15,000 psi. Three cycles of passing resulted in a liposome less than 100 nm in diameter. Trehalose was then added to the liposome to a final concentration of 10% (w/w). The resulting stable isotonic liposome was either used as liquid or lyophilized.

### Example 2

### Preparation of Liposomes Encapsulated with Lapachone

200 mg of lapachone and 6 g of L-α-Phosphatidylcholine (Soy) were dispersed in 100 mL of water using a magnetic stirrer at 200 rpm for 10 minutes at ambient temperature. The dispersed liposome (multilayer) was passed through a Microfluidic homogenizer at 15,000 psi. Three cycles of passing resulted in a liposome encapsulated with 2 mg/mL lapachone less than 100 nm in diameter. Trehalose was then added to the liposome to a final concentration of 10% (w/w). The resulting stable isotonic liposome encapsulated with lapachone was either used as liquid or lyophilized.

### Example 3

### Preparation of Liposomes Encapsulated and Micro Emulsified with Cyclosporine

500 mg of cyclosporine in 5 mL MIGLYOL and 6 g of L-α-Phosphatidylcholine (Soy) were dispersed in 100 mL of water using a magnetic stirrer at 200 rpm for 10 minutes at ambient temperature. The dispersed liposome (multilayer) was passed through Microfluidic homogenizer at 15,000 psi. Three cycles of passing resulted in a liposome encapsulated with 5 mg/mL cyclosporine less than 100 nm in diameter. Trehalose was then added to the liposome to a final concentration of 10% (w/w). The resulting stable isotonic liposome formulation encapsulated and micro emulsified with cyclosporine was either used as liquid or lyophilized.

### Example 4

### Preparation of Liposomes Encapsulated with Transferrin

200 mg of transferrin and 6 g of L-α-Phosphatidylcholine (Soy) were dispersed in 100 mL of water using a magnetic stirrer at 200 rpm for 10 minutes at ambient temperature. The dispersed liposome (multilayer) was passed through a Microfluidic homogenizer at 15,000 psi. Three cycles of passing resulted in a liposome encapsulated with 5 mg/mL transferrin less than 100 nm in diameter. Trehalose was then added to the liposome to a final concentration of 10% (w/w). The resulting stable isotonic liposome formulation encapsulated with transferrin was either used as liquid or lyophilized.

### Example 5

### Preparation of Liposomes Encapsulated with Colchicine

6 mg of sodium oleate, 10 g of trehalose and 6 g L-α-Phosphatidylcholine (Soy) were dispersed in 100 mL of water using a magnetic stirrer at 200 rpm for 10 minutes at ambient temperature. The dispersed liposome (multilayer) was passed 10 times through a Microfluidic homogenizer at 15,000 psi. 100 μL of colchicine dissolved in acetone was spiked into 1 mL of the pre-made liposomes and lyophilized. The resulting stable isotonic lyophilized liposome encapsulated with the drug is essentially free from organic solvent after lyophilization. After lyophilization, the product can be reconstituted as a 1 mg/mL, 2 mg/mL, 3 mg/mL or 4 mg/mL aqueous solution (e.g., Water For Injection).

### Example 6

### Antibody Encapsulation in Liposomes

Many therapeutic proteins, including antibodies, have a hydrophobic portion. In an embodiment, antibodies, just as other hydrophobic proteins or hydrophobic drugs, can be encapsulated by liposomes by adding the antibodies to preformed liposomes. Surprisingly, liposomal membranes were found to be capable of encapsulating the hydrophobic portions of antibodies in the alkyl chain portion of the bilayer of the liposome. This is in contrast to previous methods in which proteins including antibodies are conjugated to the lipids by chemical modification, such as via a thiol-maleimide reaction or a peptide reaction. In the present invention, no chemical modification of the therapeutic protein is required, and the protein maintains its natural structure.

HERCEPTIN is indicated for adjuvant treatment of breast cancer which is HER2 overexpressing and node positive or node negative (ER/PR negative or with one high risk feature). HERCEPTIN is provided as a sterile, white to pale yellow, preservative-free lyophilized powder for intravenous administration. Each multi-use vial of HERCEPTIN contains 440 mg trastuzumab, 400 mg α,α-1,1-trehalose dihydrate, 9.9 mg L-histidine HCl, 6.4 mg L-histidine, and 1.8 mg polysorbate 20, USP.

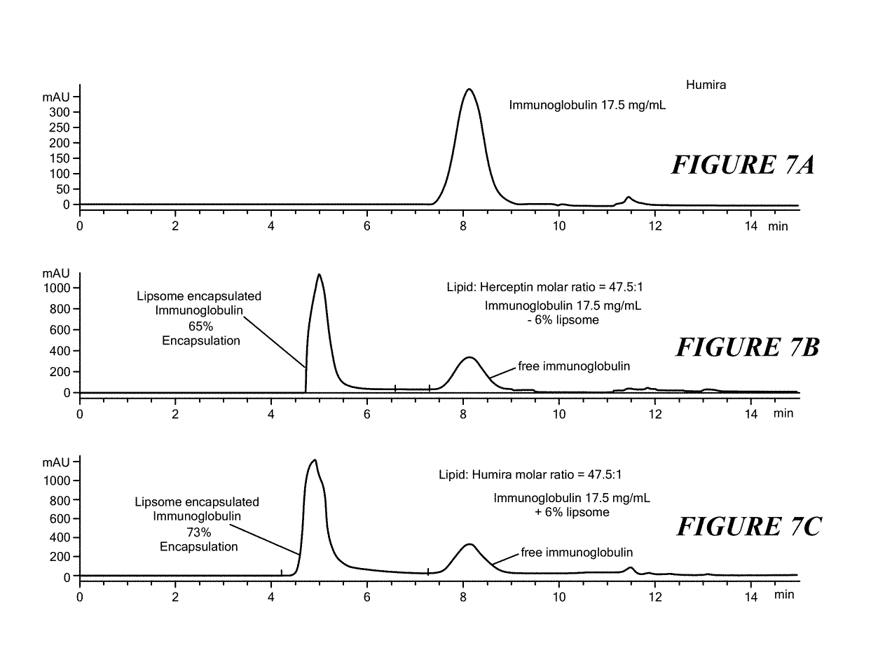

Reconstitution of a vial of HERCEPTIN with 20 mL of water for injection yielded a solution containing 21 mg/mL trastuzumab, at a pH of approximately 6.0. An aliquot of the solution containing 1.75 mg trastuzumab was lyophilized in a 3 mL vial, and was reconstituted by adding 200 μL of 6% soybean phosphatidylcholine (SPC) liposomes (prepared using microfluidization) to the lyophilized antibody at a final concentration of antibody of 8.75 mg/mL (no microfluidization was performed after addition of the antibody, just gentle mixing). The sample was analyzed by size exclusion HPLC by injecting 10 μL of sample onto a Tosoh G3000 SWXL gel filtration column using 100 mM sodium sulfate and 100 mM sodium acetate pH 6.0 as mobile phase. A HUMIRA sample without liposomes was used as control (see FIG. 7A). The data indicated that 5.6 mg/mL of trastuzumab was encapsulated in the liposome based on its elution at the void volume (FIG. 7B).

The composition of the liposomal HERCEPTIN is shown in Table 1 below.

TABLE 1
Composition of Liposomal Trastuzumab
(1 mL composition)

| Component | mg |
|---|---|
| Soy Phosphatidylcholine | 60 |
| Trehalose | 109.63 |
| Trastuzumab | 8.75 |
| Histidine HCl | 1.97 |
| Histidine | 0.13 |
| Polysorbate 20 | 0.04 |

Adalimumab is a tumor necrosis factor (TNF) blocker indicated for treatment of rheumatoid arthritis (RA), psoriatic arthritis, ankylosing spondylitis, Crohn's Disease and plaque psoriasis. Adalimumab is supplied as a sterile, preservative-free solution for subcutaneous administration. The drug product is supplied as either a single-use, prefilled pen (HUMIRA Pen) or as a single-use, 1 mL prefilled glass syringe. The solution of adalimumab is clear and colorless, with a pH of about 5.2. Each syringe delivers 0.8 mL of drug product, containing 40 mg adalimumab, 4.93 mg sodium chloride, 0.69 mg monobasic sodium phosphate dihydrate, 1.22 mg dibasic sodium phosphate dihydrate, 0.24 mg sodium citrate, 1.04 mg citric acid monohydrate, 9.6 mg mannitol, 0.8 mg polysorbate 80, and Water for Injection, USP. Sodium hydroxide is added as necessary to adjust the pH.

An aliquot containing 1.75 mg adalimumab was lyophilized in a 3 mL vial, and then reconstituted with 200 μL of 6% SPC liposome with a final concentration of 8.75 mg/mL antibody. The sample was analyzed by SEC-HPLC by injecting 10 μL of sample using Tosoh G3000 SWXL gel filtration column using 100 mM sodium sulfate and 100 mM sodium acetate pH 6.0 as mobile phase. Adalimumab sample was used as control without liposome. Surprisingly, the data indicated that 5.6 mg/mL of Herceptin was encapsulated in the liposome based on the elution at void volume (FIG. 7C). These data suggest that hydrophobic pockets of HERCEPTIN are lodged between the alkyl group of phosphatidyl choline of the bilayer without any chemical modification.

The composition of the liposomal adalimumab is shown in Table 2 below.

TABLE 2
Composition of Liposomal Adalimumab
(1 mL composition)

| Component | mg |
|---|---|
| Soy Phosphatidylcholine | 60 |
| Trehalose | 100 |
| Adalimumab | 8.75 |
| Sodium chloride | 0.216 |
| Monobasic sodium phosphate dihydrate | 0.030 |
| Dibasic sodium phosphate | 0.053 |
| Sodium citrate | 0.009 |
| Citric acid monohydrate | 0.046 |
| Mannitol | 0.420 |

Similar incorporation of antibody was obtained by adding a liquid (i.e., water-solubilized, not lyophilized) preparation of antibody to an aqueous suspension of liposomes.

### Example 7

### Preparation of Liposomes Encapsulated with Docetaxel

500 mg of docetaxel, 6 mg of sodium oleate, and 6 g of L-α-Phosphatidylcholine (Soy) was dispersed in 100 mL of water using a magnetic stirrer at 200 rpm for 10 minutes at ambient temperature. The dispersed liposome (multilayer) was passed through a Microfluidic homogenizer at 15,000 psi. Three cycles of passing resulted in liposome encapsulated with 5 mg/mL docetaxel less than 100 nm in diameter. Trehalose was then added to liposome to a final concentration of 10% (w/w). The resulting stable isotonic liposome encapsulated with docetaxel was either used as liquid or lyophilized.

### Example 8

### Preparation of Liposomes Encapsulated with Docetaxel and Transferrin

500 mg of docetaxel, 200 mg of transferrin, 6 mg of sodium oleate, and of 6 g L-α-phosphatidylcholine (Soy) were dispersed in 100 mL of water using a magnetic stirrer at 200 rpm for 10 minutes at ambient temperature. The dispersed liposome (multilayer) was passed through a Microfluidic homogenizer at 15,000 psi. Three cycles of passing resulted in liposome encapsulated with 5 mg/mL docetaxel and 2 mg/ml transferrin less than 100 nm in diameter. Trehalose was then added to the liposome to a final concentration of 10% (w/w). The resulting stable isotonic liposome encapsulated with docetaxel and transferrin is either used as liquid or it could be lyophilized. This formulation specifically targets tumors, which requires angiogenesis for its survival. Tumors are known to have transferrin receptors; incorporating transferrin in docetaxel formulations will result in less toxicity and more efficacy, as the drugs are concentrated specifically in tumors without affecting the normal cells.

### Example 9

### Preparation of Liposomes Encapsulated with Docetaxel and Lapachone

500 mg of docetaxel, 200 mg of lapachone, 6 mg of sodium oleate and 6 g L-α-phosphatidylcholine (Soy) were dispersed in 100 mL of water using a magnetic stirrer at 200 rpm for 10 minutes at ambient temperature. The dispersed liposome (multilayer) was passed through a Microfluidic homogenizer at 15,000 psi. Three cycles of passing resulted in liposome encapsulated with 5 mg/mL docetaxel and 2 mg/mL lapachone less than 100 nm in diameter.

Trehalose was then added to the liposome to a final concentration of 10% (w/w). The resulting stable isotonic liposome encapsulated with docetaxel and lapachone was either used as liquid or lyophilized. This formulation targets both nucleic acid (lapachone) and tubulin (docetaxel), thus increasing the efficacy of the treatment.

### Example 10

### Preparation of liposomes encapsulated with docetaxel, transferrin and lapachone

500 mg of docetaxel, 200 mg of transferrin, 200 mg of lapachone, 6 mg sodium oleate, and 6 g of L-α-phosphatidylcholine (Soy) were dispersed in 100 mL of water using a magnetic stirrer at 200 rpm for 10 minutes at ambient temperature. The dispersed liposome (multilayer) was passed through a Microfluidic homogenizer at 15,000 psi. Three cycles of passing resulted in liposome encapsulated with 5 mg/mL docetaxel, 2 mg/mL transferrin and 2 mg/mL lapachone less than 100 nm in diameter. Trehalose was then added to the liposome to a final concentration of 10% (w/w). The resulting stable isotonic liposome encapsulated with docetaxel, lapachone and transferrin was either used as liquid or lyophilized. This formulation specifically targets tumors, which require angiogenesis for their survival. Tumors are known to have transferrin receptors; incorporating transferrin in docetaxel formulations will result in less toxicity and more efficacy, as the drugs are concentrated specifically in tumors without affecting the normal cells. This formulation will further target both nucleic acid (lapachone) and tubulin (docetaxel), thus increasing the efficacy of the therapy as well.

### Example 11

### Solubilization of Docetaxel in Oleic Acid

Docetaxel (5 mg) was added to 31 μL of oleic acid and mixed using a VORTEX. Ethanolamine (6 μL) was subsequently added and mixed using a VORTEX. After mixing, the sample was dispersed in 963 μL of water for injection. Docetaxel was soluble and the formulation clear at 5 mg/mL. Docetaxel remained soluble, and the formulation was stable, upon dilution (1:10 in 2.5% glycerol) up to 24 hours as analyzed by a reverse phase high performance liquid chromatography method.

### Example 12

### Solubilization of Docetaxel in Sodium Oleate

Docetaxel (5.0 mg) was added to 1 mL of 0.05 M sodium oleate and mixed using a VORTEX. Docetaxel was soluble and the formulation clear at 5.0 mg/mL. Docetaxel remained soluble, and the formulation was stable, upon dilution (1:10 in 2.5% glycerol) up to 24 hours as analyzed by a reverse phase high performance liquid chromatography method.

### Example 13

### Solubilization of Docetaxel in Sodium Caprylate

Docetaxel (2 mg) was added to 1 mL of 1 M sodium caprylate and mixed by a VORTEX. Docetaxel was soluble and the formulation clear at 2 mg/mL. Docetaxel remained soluble, and the formulation was stable, upon dilution (1:2 2.5% glycerol) up to 24 hours as analyzed by a reverse phase high performance liquid chromatography method.

### Example 14

### Solubilization of Docetaxel in Sodium Linoleate

Docetaxel (7.0 mg) was added to 1 mL of 1 M sodium linoleate and mixed using a VORTEX. Docetaxel was soluble and the formulation clear at 7.0 mg/mL. Docetaxel remained soluble, and the formulation was stable, upon dilution (1:10 2.5% glycerol) up to 24 hours as analyzed by a reverse phase high performance liquid chromatography method.

### Example 15

### Solubilization of Docetaxel in Oleate Liposome

Docetaxel (5 mg) was added to 1 mL of pre-made liposome containing 2 mM sodium oleate and 6% soy phosphatidyl choline, 10% trehalose, pH adjusted to 5-7 using acetic acid, mixed using a VORTEX, and microfluidized using a MICROFLUIDIZER with 10 passes at 15,000 psi. Five additional passes with the MICROFLUIDIZER at 15,000 psi were made to incorporate all 5 mg of the drug into 1 mL of pre-made liposome. Docetaxel was soluble and the formulation was transparent at 5 mg/mL. Docetaxel remained soluble, and the formulation was stable, upon dilution (1:10 in D5W) up to 48 hours as analyzed by a reverse phase high performance liquid chromatography method. The formulation is lyophilizable and the lyophilized formulation is stable for more than a year.

### Example 16

### Solubilization in Fatty Acid Dissolved in Organic Solvents

Hydrophobic or lipophilic compounds can be solubilized directly in sodium (or any ion) salt of fatty acid dissolved in an organic solvent. The organic solvent is subsequently removed using rotary evaporation, spray drying, or any other pharmaceutically acceptable processes. The drug-sodium salt of fatty acid mixture is subsequently dispersed in water, isotonic glycerol, or any pharmaceutically acceptable isotonic solution to achieve a desired concentration of the solubilized compound. For example, docetaxel (5 mg/mL) was dissolved in 100 mM sodium oleate in 100% ethanol. Ethanol was completely evaporated using rotary evaporation at 20° C. The docetaxel-sodium oleate was reconstituted with Water For Injection (“WFI”) or 2.5% glycerol to provide 5 mg/mL, 10 mg/mL, or 20 mg/mL concentration of docetaxel. The reconstituted docetaxel remained in solution for 2 hours, 4 hours, 6 hours, 8 hours or 24 hours.

### Example 17

### Solubilization of Drug in Organic Solvent and Injection into Nanosome-Oleate

50 μL of Docetaxel in ethanol (100 mg/mL) was added to 1 mL of pre-made liposome containing 2 mM sodium oleate and 6% soy phosphatidyl choline, 10% trehalose and pH adjusted to 5-7 using acetic acid, mixed using a VORTEX and microfluidized using a Microfluidizer with 10 passes at 15,000 psi. Docetaxel was soluble and the formulation is transparent at 5 mg/mL. Docetaxel remained soluble, and the formulation was stable, upon dilution (1:10 in D5W) up to 48 hours as analyzed by a reverse phase high performance liquid chromatography method. The formulation is lyophilizable and the lyophilized formulation is stable for more than a year. The lyophilized product is reconstituted with water for injection at 5 mg, 10, or 20 mg/ml concentration, essentially free of organic solvent.

### Example 18

### Safety of MIRADOCETAXEL in Nanosome Formulation

Safety of docetaxel formulated as in Example 15 was studied using a nude mouse model to understand the maximum tolerable dose for MIRADOCETAXEL as compared to TAXOTERE. The maximum tolerated dose (“MTD”), defined as the highest dose of a drug or treatment that does not cause unacceptable side effects, is determined in clinical trials by testing increasing doses on different groups of people until the highest dose with acceptable side effects is found.

The MTD of TAXOTERE and MIRADOCETAXEL was addressed in nude mice. In the mice model, MTD is considered the highest dose which does not kill any mice in a group or does not cause 20% weight loss. The drugs were injected to group of 5 nude mice by I.V. at different concentrations. The injection schedules were Q7D3 (3 injections, one every 7 days). The percent weight loss and gross examination of internal organ were monitored for each animal.

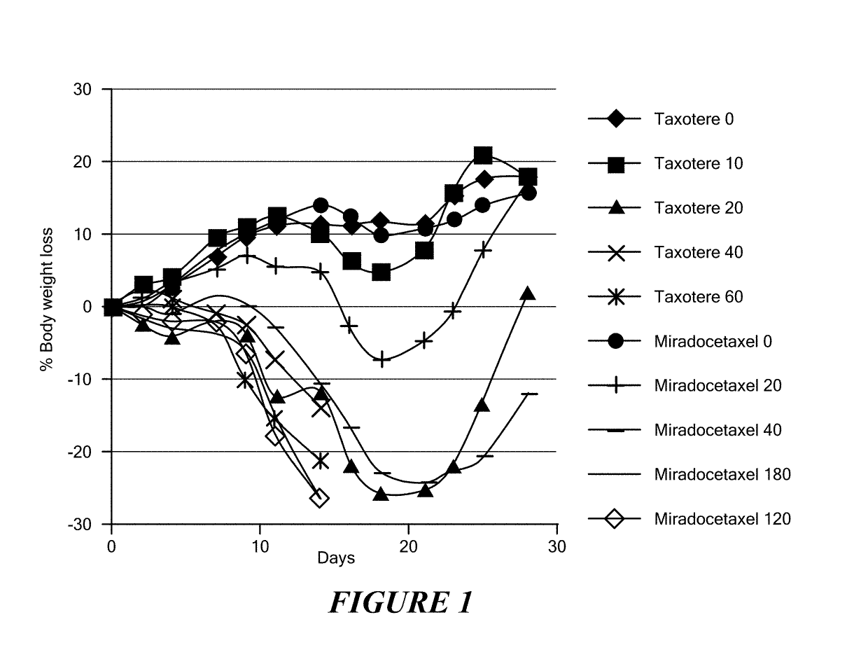

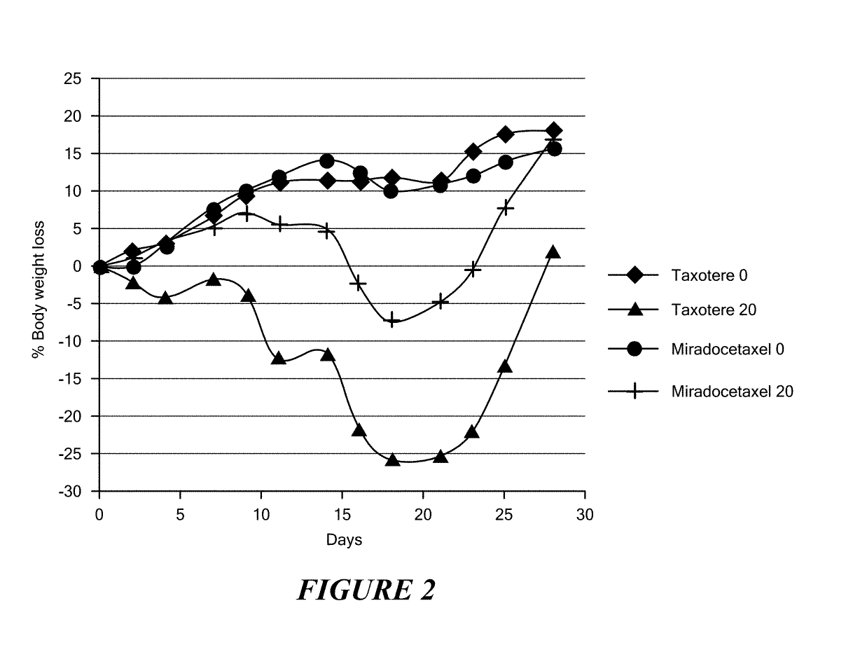

The maximum tolerable doses in athymic nude mice for TAXOTERE and MIRADOCETAXEL are presented in Table 3 and percent body weight losses are presented in FIGS. 1 and 2.

TABLE 3

| Drug | MTD (mg/kg) |
|---|---|
| MIRADOCETAXEL | 30 |
| TAXOTERE | 15 |

The weight loss at 20 mg/mL concentration was more than 20% for the TAXOTERE injection group, while less than 10% weight loss was noticed for the MIRADOCETAXEL injection group (FIG. 2). The gross examination of organs suggested no damage in any group. Based on this MTD result MIRADOCETAXEL is considered safer than TAXOTERE.

### Example 19

### Efficacy of Docetaxel Formulated in a Fatty Acid Salt Nanosome Using Xenograft of Human Melanoma Tumor and Prostate Tumor

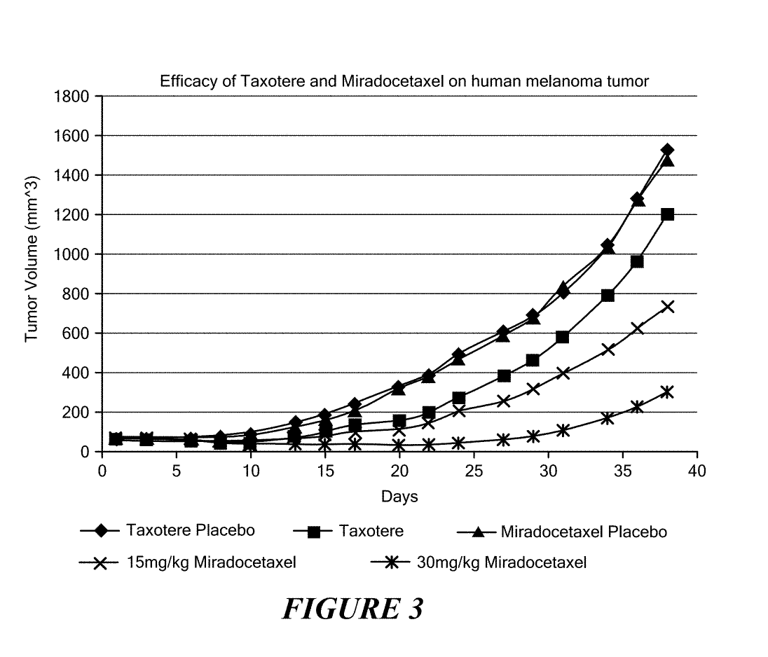

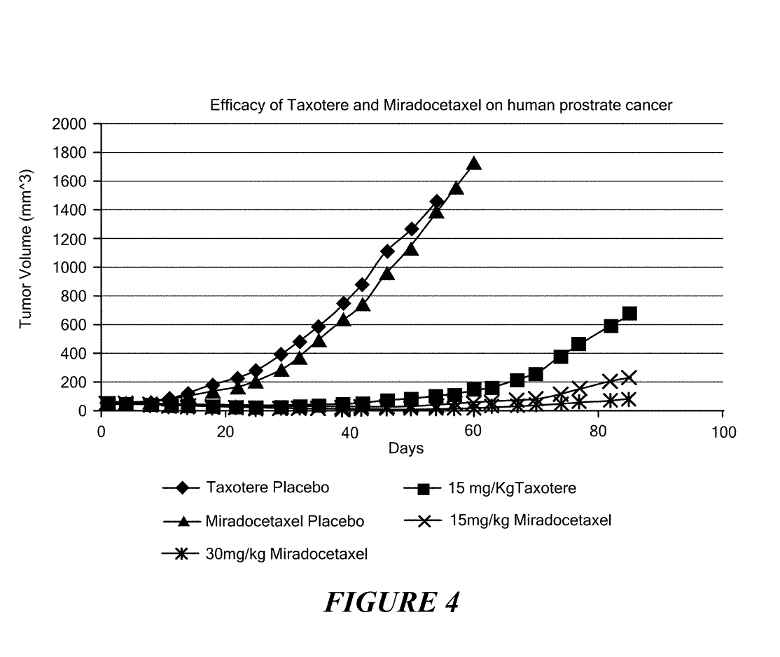

Efficacy of docetaxel formulated as in Example 17 was studied using the xenograft of human melanoma tumor and prostrate tumor. Two separate in-vivo studies were conducted each employing a different cancer cell line xenografted into mice. Athymic mice (nu/nu) implanted with either human melanoma tumor A375 or prostate tumor PC 3 cells and the cells were allowed to establish tumors. The mice were then treated with TAXOTERE or MIRADOCETAXEL (Q7DX3) (3 injections, one every 7 days). TAXOTERE was delivered as a Tween 80-ethanol-saline formulation (15 mg/kg) and MIRADOCETAXEL was delivered as sodium oleate-liposome formulation at concentrations of 15 mg/kg and 30 mg/kg. As shown in Example 18 above, MIRADOCETAXEL delivery was shown to reduce toxicity as compared to TAXOTERE. See FIGS. 1 and 2. This justified the use of the higher dose of 30 mg/kg for MIRADOCETAXEL. The administration of 15 mg/kg of MIRADOCETAXEL was more efficacious than the administration of 15 mg/kg TAXOTERE, as evidenced by tumor growth delay in both the tumor models. See FIGS. 3 and 4. Tumor growth inhibition (T/C) is the average tumor size of the treated groups (T) divided by the average tumor size of the control group (C) at a time when the average tumor size in the control group has reached approximately 1500 mm³. A T/C value equal to or less than 42% is considered significant antitumor activity by the Drug Evaluation Branch of the Division of Cancer Treatment, National Cancer Institute (NCI). Tumor growth delay (T-C) is the difference between the average time, in days, required for the treatment group tumor (T) to reach approximately 250 mm³, and the average time, in days, for the control group tumor (C) to reach the same size. The results obtained on T/C, T-C for are presented in Tables 2 and 3. The data indicates, that TAXOTERE has no antitumor activity against melanoma, as T/C value was more than 42%. However, MIRADOCETAXEL treatment has significant tumor inhibition with the T/C value of 20. Moreover, the tumor growth delay (T-C) results showed that MIRADOCETAXEL treatment delays the growth of tumor to a greater degree than TAXOTERE.

TABLE 4
A375 Human Melanoma Tumor Response to Treatments

| | Tumor Load | T/C | 250 mm³ | T-C |
|---|---|---|---|---|
| Group | (mm³ day 38) | (%) | (Day #) | (Days) |
| 13% Ethanol | 1527 | 100 | 18 | 0 |
| 15 mg/kg TAXOTERE | 1203 | 79 | 23 | 5 |
| MIRADOCETAXEL*** placebo | 1473 | 96 | 18 | 0 |
| 15 mg/kg MIRADOCETAXEL | 732 | 48 | 27 | 9 |
| 30 mg/kg MIRADOCETAXEL | 299 | 20 | 37 | 19 |

*T/C: Tumor growth inhibition.
***All MIRADOCETAXEL formulations were reconstituted in water.

TABLE 5
PC 3 Human Prostate Tumor Response to Treatments

| | Tumor Load | T/C | 250 mm³ | T-C |
|---|---|---|---|---|
| Group | (mm³ day 38) | (%) | (Day #) | (Days) |
| 13% Ethanol | 1458 | 100 | 23 | 0 |
| 15 mg/kg TAXOTERE | 90.9 | 6.23 | 69 | 46 |
| MIRADOCETAXEL*** placebo | 1387.6 | 100 | 26 | 0 |
| 15 mg/kg MIRADOCETAXEL | 34.5 | 2.48 | 86 | 60 |
| 30 mg/kg MIRADOCETAXEL | 11.35 | 0.82 | 98 | 72 |

*T/C: Tumor growth inhibition.
**T-C: Tumor growth delay. Time to achieve 250 mm3 in the respective control group “C” was 23 and 26 days.
***All MIRADOCETAXEL formulations were reconstituted in water.

### Example 20

### Pharmacokinetic of Docetaxel Formulated in a Fatty Acid Salt Nanosome Using Rat Model System

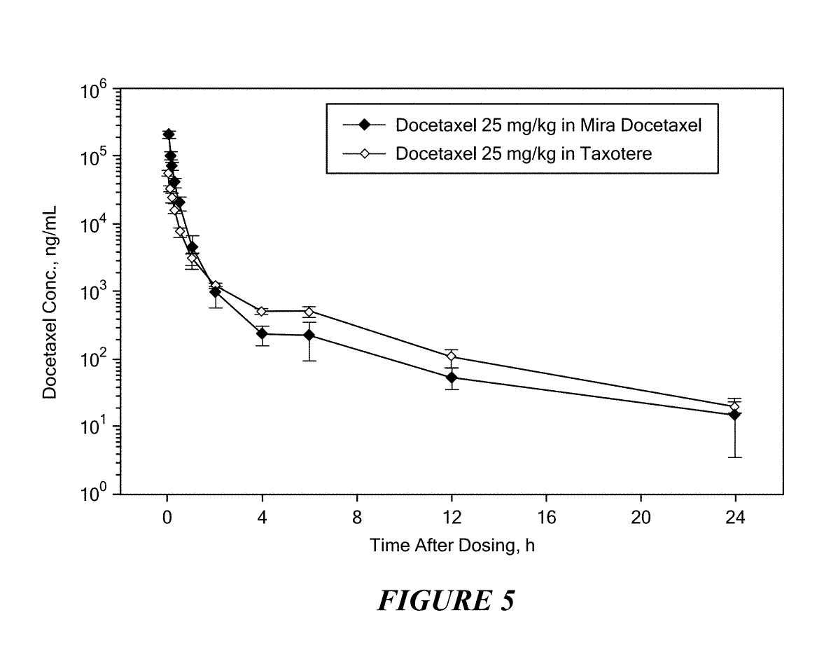

The pharmacokinetics of docetaxel in male rats following a single intravenous dosing of 25 mg/kg docetaxel in TAXOTERE or MIRADOCETAXEL formulation was evaluated. TAXOTERE displayed a multi-exponential decay with harmonic mean T_{1/2} values of about 4 to 5 hours. MIRADOCETAXEL resulted in a 2.2-fold higher plasma docetaxel exposure (AUC(0-inf)) and about 2-fold lower systemic CL than the corresponding pharmacokinetics of docetaxel TAXOTERE following dosing as shown in Table 6 and FIG. 5.

The plasma concentrations of docetaxel displayed the characteristics of a multi-exponential curve with harmonic mean T_{1/2} of 4.02+0.266 hours (Table 6 and FIG. 5). Docetaxel in TAXOTERE had AUC(0-inf), CL and Vₛₛ mean values were 21,100±1290 ngh/mL, 1.19±0.0729 L/h/kg and 2.35+0.0503 L/kg, respectively (Table 6). Docetaxel in MIRADOCETAXEL, AUC(0-inf), CL and Vₛₛ mean values were 46,500±7640 ngh/mL, 0.548±0.0953 L/h/kg and 0.408±0.176 L/kg, respectively (Table 6).

TABLE 6
Comparison of Docetaxel PK Parameters in Male Rats
Following a Single 25 mg/kg Intravenous Dose of
Docetaxel in TAXOTERE or MIRADOCETAXEL

| | TAXOTERE | | MIRADOCETAXEL | |
|---|---|---|---|---|
| Parameter | Mean | SD | Mean | SD |
| T_{1/2}, h^{b} | 4.02 | 0.266 | 4.73 | 1.47 |
| AUC(0-inf), | 21,100 | 1,250 | 46,500 | 7,640 |
| ng h/mL | | | | |
| AUC(0-inf), | 21.1 | 1.25 | 46.5 | 7.64 |
| μg · h/mL | | | | |
| Vₛₛ, L/kg | 2.35 | 0.0503 | 0.408 | 0.176 |
| CL, L/h/kg | 1.19 | 0.0729 | 0.548 | 0.0953 |

^{a}Docetaxel AUC(0-inf) following MIRADOCETAXEL/docetaxel AUC(0-inf) following TAXOTERE
^{b}Docetaxel CL following MIRADOCETAXEL/docetaxel CL following TAXOTERE.

### Example 21

### Unit Dosage Forms for MIRADOCETAXEL

MIRADOCETAXEL is prepared as a lyophilized powder in vials of suitable size. A desired dosage can be filled in a suitable container and lyophilized to obtain a powder containing essentially fatty acid salt, phospholipid and docetaxel in the desired quantity. Such containers are then reconstituted with sterile aqueous diluent to the appropriate volume at the point of use to obtain a homogeneous clear solution of docetaxel in the diluent. This reconstituted solution can be directly administered to a patient either by injection or infusion with standard i.v. infusion sets.

### Example 22

### Stability of Lyophilized Liposome-Encapsulated MIRADOCETAXEL

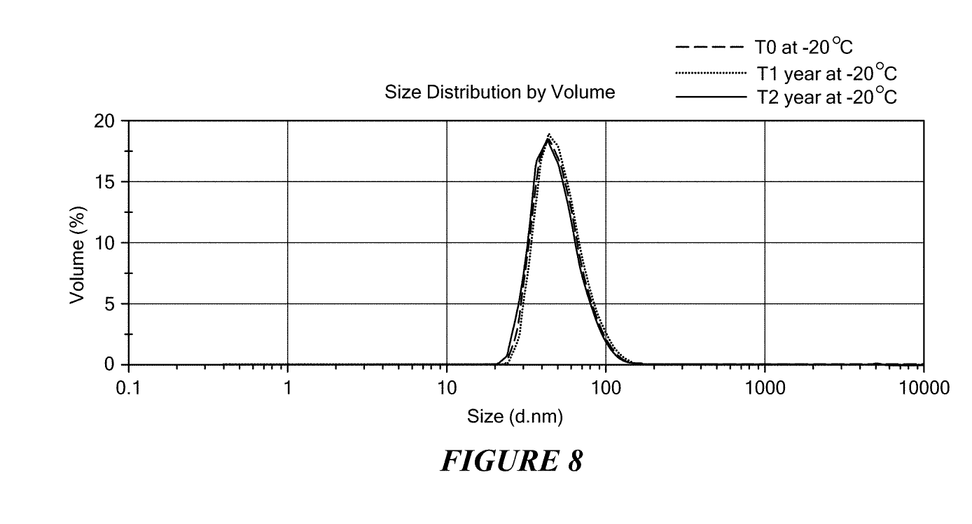

The stability of lyophilized liposomes encapsulating MIRADOCETAXEL was evaluated at high (40° C.) and low (−20° C.) temperatures. As shown in Table 7, after 6 months at 40° C. the concentration, purity, and visual appearance of the formulation were not changed, and particle size remained less than 70 nm. The results indicate that the formulation is stable for at least for 2 years at room temperature. FIG. 8 shows the initial particle sizes, as well as particle sizes after 1 year and after 2 years of storage at −20° C., showing that the average particle size remained about 62.9-64.4 nm after 2 years of storage.

What is claimed is:

1. A method of incorporating a hydrophobic therapeutic agent into preformed liposomes, the method comprising the steps of:
(a) providing (i) a liposome suspension comprising a plurality of preformed liposomes suspended in an aqueous medium, the liposomes comprising lipid forming a lipid bilayer phase, and (ii) a solid form of a hydrophobic therapeutic agent;
(b) adding the solid form of the hydrophobic therapeutic agent to the liposome suspension, thereby forming a liposome-drug suspension; and
(c) homogenizing the liposome-drug suspension;
whereby the hydrophobic therapeutic agent is incorporated into the lipid bilayer phase; and
wherein step (c) is performed at a temperature at or below ambient temperature.

2. The method of claim 1, wherein steps (b) and/or (c) are performed in the absence of solvent.

3. The method of claim 1, wherein steps (b) and/or (c) are performed in the absence of surfactant.

4. The method of claim 1, wherein the hydrophobic therapeutic agent is a small molecule drug or an antibody.

5. The method of claim 1, wherein at least about 80% of the hydrophobic therapeutic agent is bound to the lipid bilayer phase after step (c).

6. The method of claim 1, wherein the molar ratio of therapeutic agent to liposomal lipid is at least about 1:10.

7. The method of claim 1, wherein homogenization of the liposome-drug suspension in step (c) is performed by microfluidization, sonication, extrusion, freeze-thaw, or a combination thereof.

8. The method of claim 1, wherein the therapeutic agent does not contact solvent during steps (b) and (c), and does not contact the liposomal lipid prior to the formation of the liposomes.

9. The method of claim 1, wherein the liposomes are essentially unilamellar after step (c).

10. The method of claim 1, wherein the liposomes have a diameter of about 100 nm or less after step (c).

11. The method of claim 1, wherein the liposomes have a diameter of about 50 nm or less after step (c).

12. The method of claim 1, wherein the liposome suspension provided in step (a) comprises an additional therapeutic agent present in the aqueous medium and/or the liposomes.

13. The method of claim 1, wherein the composition of the aqueous medium inside and outside the liposomes is identical in steps (a), (b), and (c).

14. The method of claim 1, wherein the liposomal lipid comprises not more than 20% saturated fatty acids.

15. The method of claim 1, wherein the liposomal lipid comprises L-α-phosphatidylcholine.

16. The method of claim 1, wherein the method further comprises, after step (c), performing sterile filtration, lyophilization, or lyophilization and reconstitution with an aqueous medium.

17. The method of claim 1, wherein the therapeutic agent remains bound to the lipid bilayer phase of the liposomes after step (c) for at least 2 months upon storage in the aqueous medium at about 4° C.

18. The method of claim 1, wherein the therapeutic agent remains associated with the lipid bilayer phase after step (c) followed by lyophilization and storage for at least 1 year at ambient temperature and reconstitution in an aqueous medium.

19. The method of claim 1, wherein average liposome size after step (c) remains less than about 100 nm for at least 2 months upon storage in the aqueous medium at about 4° C.

20. The method of claim 1, wherein after step (c) followed by lyophilization, storage for at least 1 year at ambient temperature, and reconstitution in an aqueous medium, the average liposome size remains less than about 100 nm.

21. The method of claim 1, wherein said temperature is from about 15° C. to about 35° C.

22. The method of claim 1, wherein the lipid bilayer phase is liquid crystalline at said temperature.

23. The method of claim 1, wherein the lipid concentration of the liposome-drug suspension in steps (b) and (c) is from about 1 to about 7% by weight.

24. The method of claim 1, wherein the solid hydrophobic therapeutic agent added in step (b) provides a total concentration of the agent in the liposome-drug suspension of from about 1 to about 20 mg/mL.

25. A method of incorporating a hydrophobic therapeutic agent into preformed liposomes, the method comprising the steps of:
(a) providing (i) a liposome suspension comprising a plurality of preformed liposomes suspended in an aqueous medium, the liposomes comprising lipid forming a lipid bilayer phase, and (ii) a therapeutic agent concentrate comprising a hydrophobic therapeutic agent dissolved in a liquid medium comprising or consisting of solvent;
(b) adding the therapeutic agent concentrate to the liposome suspension to form a liposome-drug suspension, wherein the total concentration of solvent in the liposome-drug suspension is not more than 10 weight percent; and
(c) homogenizing the liposome-drug suspension;
whereby the hydrophobic therapeutic agent is incorporated into the lipid bilayer phase; and
wherein step (c) is performed at a temperature at or below ambient temperature.

26. The method of claim 25, wherein the total concentration of solvent in the liposome-drug suspension in step (b) is not more than about 5 weight percent.

27. The method of claim 25, wherein the solvent is a water miscible organic solvent.

28. The method of claim 25, wherein the liquid medium further comprises water or an aqueous medium.

29. The method of claim 25, wherein the solvent is selected from the group consisting of alcohols, ketones, ethers, organic acids, organic bases, and mixtures thereof

30. The method of claim 25, wherein the solvent is selected from the group consisting of ethanol, propanol, isopropanol, butanol, isobutanol, and DMSO.

31. The method of claim 25, wherein the liquid medium is 100% ethanol.

32. The method of claim 25, wherein step (c) is performed without the use of surfactant.

33. The method of claim 25, wherein the hydrophobic therapeutic agent is a small molecule drug or an antibody.

34. The method of claim 25, wherein at least about 80% of the hydrophobic therapeutic agent is associated with the lipid bilayer phase after step (c).

35. The method of claim 25, wherein the molar ratio of therapeutic agent to lipid is at least about 1:10.

36. The method of claim 25, wherein homogenization of the liposome-drug suspension in step (c) is performed by microfluidization, sonication, extrusion, freeze-thaw, or a combination thereof.

37. The method of claim 25, wherein the therapeutic agent does not contact the liposomal lipid prior to the formation of the liposomes.

38. The method of claim 25, wherein the liposomes are essentially unilamellar after step (c).

39. The method of claim 25, wherein the liposomes have a diameter of about 100 nm or less after step (c).

40. The method of claim 25, wherein the liposomes have a diameter of about 50 nm or less after step (c).

41. The method of claim 25, wherein the liposome suspension provided in step (a) comprises an additional therapeutic agent present in the aqueous medium and/or the liposomes.

42. The method of claim 25, wherein the composition of the aqueous medium inside and outside the liposomes is identical in steps (a), (b), and (c).

43. The method of claim 25, wherein the liposomal lipid comprises not more than 20% saturated fatty acids.

44. The method of claim 25, wherein the liposomal lipid comprises L-α-phosphatidylcholine.

45. The method of claim 25, wherein the method further comprises, after step (c), performing sterile filtration, lyophilization, or lyophilization and reconstitution with an aqueous medium.

46. The method of claim 25, wherein the therapeutic agent remains bound to the lipid bilayer phase of the liposomes after step (c) for at least 2 months upon storage in the aqueous medium at about 4° C.

47. The method of claim 25, wherein the therapeutic agent remains associated with the lipid bilayer phase after step (c) followed by lyophilization and storage for at least 1 year at ambient temperature and reconstitution in an aqueous medium.

48. The method of claim 25, wherein average liposome size after step (c) remains less than about 100 nm for at least 2 months upon storage in the aqueous medium at about 4° C.

49. The method of claim 25, wherein after step (c) followed by lyophilization, storage for at least 1 year at ambient temperature, and reconstitution in an aqueous medium, the average liposome size remains less than about 100 nm.

50. The method of claim 25, wherein said temperature is from about 15° C. to about 35° C.

51. The method of claim 25, wherein the lipid bilayer phase is liquid crystalline at said temperature.

52. The method of claim 25, wherein the lipid concentration of the liposome-drug suspension in steps (b) and (c) is from about 1 to about 7% by weight.

53. The method of claim 25, wherein the solid hydrophobic therapeutic agent added in step (b) provides a total concentration of the agent in the liposome-drug suspension of from about 1 to about 20 mg/mL.

54. The method of claim 25, further comprising:
(d) lyophilizing the homogenized liposome suspension, whereby said solvent is removed.
